SEQUENCE LISTING (19 sequences)

SEQ ID NO: 1 - DNA, 576 bp, synthetic construct
  misc_feature 1..576: note = TPD coding sequence
  source 1..576: mol_type = other DNA
gaccccggtttcggcccgcatcagcgactggagaagctcgattctctcctttccgactacgatattttgtccctgtcaaacattcaacagcactccgtaaggaaacgcgacttgcaaactagcacgcatgtcgaaaccctcttgactttctcagctctgaaaaggcacttcaaactctacctcacatcttctacggaacggttctcccaaaacttcaaagtagtcgttgttgacgggaaaaacgaatcagagtatacggtgaagtggcaggactttttcaccggccatgtcgtcggagaaccagacagccgcgtactcgcacacatccgcgacgacgacgtcatcattcgcatcaataccgatggagccgaatataacatcgaaccactttggaggttcgttaacgacactaaagacaagagaatgctggtctataagtcagaagatattaaaaacgtttcccggctccagtcccccaaagtgtgcgggtacctcaaggtggataacgaagaacttttgcctaaagggctcgtagatcgcgaacccccagaggagctggttcatagagtaaagcgg

SEQ ID NO: 2 - protein, 192 aa, synthetic construct
  REGION 1..192: note = TPD amino acid sequence
  VARIANT 34: note = Xaa is Arg or can be replaced by the following AA: Alanine, Asparagine, Aspartic Acid, Cysteine, Glutamine, Glutamic Acid, Glycine, Histidine, Isoleucine, Leucine, Methionine, Phenylalanine, Proline, Serine, Threonine, Tryptophan, Tyrosine
  VARIANT 162: note = Xaa is Cys or can be replaced by alanine, or valine.
  VARIANT 189: note = Xaa is Arg or can be replaced bythe following AA: Alanine, Asparagine, Aspartic Acid, Cysteine, Glutamine, Glutamic Acid, Glycine, Histidine, Isoleucine, Leucine, Methionine, Phenylalanine, Proline, Serine, Threonine, Tryptophan, Tyrosine
  VARIANT 192: note = Xaa is Arg or can be replaced by the following AA: Alanine, Asparagine, Aspartic Acid, Cysteine, Glutamine, Glutamic Acid, Glycine, Histidine, Isoleucine, Leucine, Methionine, Phenylalanine, Proline, Serine, Threonine, Tryptophan, Tyrosine
  VARIANT 36: note = Xaa is Arg or can be replaced by the following AA: Alanine, Asparagine, Aspartic Acid, Cysteine, Glutamine, Glutamic Acid, Glycine, Histidine, Isoleucine, Leucine, Methionine, Phenylalanine, Proline, Serine, Threonine, Tryptophan, Tyrosine
  VARIANT 35: note = Xaa is Lys or can be replaced by the following AA: Alanine, Asparagine, Aspartic Acid, Cysteine, Glutamine, Glutamic Acid, Glycine, Histidine, Isoleucine, Leucine, Methionine, Phenylalanine, Proline, Serine, Threonine, Tryptophan, Tyrosine
DPGFGPHQRLEKLDSLLSDYDILSLSNIQQHSVXXXDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVXGYLKVDNEELLPKGLVDREPPEELVHXVKX

SEQ ID NO: 3 - DNA, 624 bp, synthetic construct
  misc_feature 1..624: note = SSPH1 coding sequence
  source 1..624: mol_type = other DNA
atgtttaatatccgcaatacacaaccttctgtaagtatgcaggctattgctggtgcagcggcaccagaggcatctccggaagaaattgtatgggaaaaaattcaggtttttttcccgcaggaaaattacgaagaagcgcaacagtgtctcgctgaactttgccatccggcccggggaatgttgcctgatcatatcagcagccagtttgcgcgtttaaaagcgcttaccttccccgcgtgggaggagaatattcagtgtaacagggatggtataaatcagttttgtattctggatgcaggcagcaaggagatattgtcaatcactcttgatgatgccgggaactataccgtgaattgtcaggggtacagtgaagcacatgacttcatcatggacacagaaccgggagaggaatgcacagaattcgcggagggggcatccgggacatccctccgccctgccacaacggtttcacagaaggcagcagagtatgatgctgtctggtcaaaatgggaaagggatgcaccagcaggagagtcacccggccgcgcagcagtggtacaggaaatgcgtgattgcctgaataacggcaatccagtgcttaacgtgggagcgtcaggtcttacc

SEQ ID NO: 4 - DNA, 18 bp, synthetic construct
  misc_feature 1..18: note = HIS Tag coding sequence
  source 1..18: mol_type = other DNA
catcaccatcaccatcac

SEQ ID NO: 5 - DNA, 3151 bp, Homo sapiens
  source 1..3151: mol_type = genomic DNA
agcggcggccggaagcggcgagccggccggaacgccaccgcaccgggggcgcgagccggcggagaaccccagaggcggcgcgggagggaaaagaggagaggggcaggccgggcggacccgcccccccgaggagcagccgaaaccccgcaggcgaaggcgcccagagaggggagcggagcggggccgggaacagaggcagccccaccgaccagcgggcccggcggcgccgcgaccccggagacccgggccggcccccaccagagaccgagaagcgacgcccagacacgaacccacaaaccagcagcacggaagaaaaagagacacagaccaacacagagaaacacacaaccagcgaaaaggcaaaaaaccgacacaagacgaacgcacaaaacaaggcgggggggaggaaaaacgaaagcgagacacgaaaaggcaggacccacggacacggggggagccgaccagggcagcccacaaagagagagagaaacagaacaacacagaggggccgaaaaacaagagccacggagagaagaaccaaagacaaaagaagagaaaacgaagaacaagaagcacggcagcccaaaagggggaaaaagggaaagaagaggccccaaaagggagagacagagaaccaccgaagagcgcacgaggaaaagaagagcgacccagacccagaagaacacggaaaaggggagcagacacgccacagaacagggcagaggggaagagagacaacacaaaacaaagagcaagacagaggagacacacggaacaccagggaaagcaggaaaggcaggaaacagaagagcagacgcaccaagcccacaagaggaaaaccgggaaaagcacacaacaggcaaaaagacccaaagaagaaaaggagcgggaggaagaggcagagcaaagcgaaagcgaggaagcacaaaggcggcacacccacaaccaagagaagggaaccggaagcagggcccccagagcaaacagccaggaggggccaaaggcaaagcccaggggaagaaaaaacagaaaggggacgagcacaaagaaaggaaaaccaccacaaaggaagcgaccggacaaccagaagggacaaaggagcagaacagaccggaggcagcagaaggccccgaagaggaccagggagggaaaagcagacccaagcggagggcgacacgagaacaaaagagcaaacgcagaaacaacaacaaagaccagaaagaaggcccaggaggcaagaacgcagcaaaaagggggaaccgagggggagaaggagaagagggaccggcacagacgaacaacgacaccgcgcaacagcgacgcacggaaggaagggccaggcaggacaggaacagccgcgaaaaacgcaggagacgcccagaagaaggccaggaggcgaaagcacgcaaaggcggccacgcacaggaaagcaggaggcccgccccaggaaagcgaagagacacggcggacggcaaggaaggagggaaagcaccccgcgagagggaacagcagcggagccggcagaagaaacgacaacccgcaaggggcgcagggacccggccgcgggcccagcgagcgaacaaaagaacagaggaaaggaaagcccgacagaggaggacagaaggcaaaggagaaacgagacaggagaagaacgagggacagaccagcgagcacaaacggaaagagcagacaacacggggcgccggcccgaaggacccagcacgccaggggaaagaaaggaaaacagagaaccgccgcaccccagaacgcgaaagcgagcagcaggacgcacggcgcaacaaacccccgcgccccagacccaggccgccgcagccgccccggaccccggcgccagcagcccaaaacggaccaccagagaaggacaccaccaggaagaccccagcacagaccacaaggacgaggaggggagaaggacccccccaaaagcagcacagcgccaagcagaggaccacggaccaccggcaccagaaggaaaaggcgccccaaacgcagcgcagaacgggacagcaaagaaacagaggcaaagccagcccgacaagggcaaaaaaagagaccacaaacaacacagcgaggaagacgggaaggacagcagagcggcagggaacccgcaggaaacagcggggggccccccgaaaaggaagggaagggaacagcagaggccaggagaaaaacgaccggggcaaaagcagaaaaacagcggagggagaaaacggaaaaacaagaaacagcacgacagggaaagacagaacacgaagaaccgcgaaagaggcaggcaaggagacaacggaaaggcccaacaaaaacaaaggagaaaacagaacagccaaaggggcacagaacccagagagagcagggaaaaaaacaaaagaaacagggaaaggcccaaacaagacaagcccagaaaccaaccaccaccacaggagggcaacaggagccaggaaaggaacaccaacgagggcccgagcgggcagcgaaaagaccagacccgacgaagggaacagccagaaaacacaggcgggagggggccagccgaacccagcacggagaggcgagggcgccacacacccagccggggacagaggagaccgcccaaaaaaaaaaaaaaaaaaaaaaaacacacacaaaccagaaaaaaaacaaagagccgcaaacacagaaaagaggggagaacaacaacaacaacaacaaaaaaaacaggggaaggagagccagccaggagaagaaacaccgcccaggggggggcgccccacaagacgaaccagaaacccagacccacaagcagaagaaaccacagcccaggacggagggcgcaacgggaaggaaaaaacaagccggacgacaaaagccaaaaagcccccaagaacgaaaggccaccagccaaaacaaacgaaagagggcagaaccaaaccggacaacaacccccaccccccaccaaggacagaccagaacgaacaaacgaacgaccaggagcacaacagcagaacaacagacacggaagaacaagggagggggggggggagagaggaaacagccaaaagaagaagaaaaaccaaaaaaggacaaaaacaagcgcaaagaaaacaagcgccagggaaaagcgagcacgaacgcaaaaaaacgcgaaacacaa

SEQ ID NO: 6 - protein, 824 aa, Homo sapiens
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTVKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAEDDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVTRSEKAASFKLQRQNRVDSKETEC

SEQ ID NO: 7 - protein, 208 aa, synthetic construct
  REGION 1..208: note = SSPH1 amino acid sequence
MFNIRNTQPSVSMQAIAGAAAPEASPEEIVWEKIQVFFPQENYEEAQQCLAELCHPARGMLPDHISSQFARLKALTFPAWEENIQCNRDGINQFCILDAGSKEILSITLDDAGNYTVNCQGYSEAHDFIMDTEPGEECTEFAEGASGTSLRPATTVSQKAAEYDAVWSKWERDAPAGESPGRAAVVQEMRDCLNNGNPVLNVGASGLT

SEQ ID NO: 8 - protein, 30 aa, synthetic construct
  REGION 1..30: note = signal peptides
MPLSVGQGYFTSSISSEKFNAIKESARLPE

SEQ ID NO: 9 - protein, 30 aa, synthetic construct
  REGION 1..30: note = signal peptides
MLKYEERKLNNLTLSSFSKVGVSNDARLYI

SEQ ID NO: 10 - protein, 30 aa, synthetic construct
  REGION 1..30: note = signal peptides
MPVTLSFGNRHNYEINHSRLARLMSPDKEE

SEQ ID NO: 11 - protein, 30 aa, synthetic construct
  REGION 1..30: note = signal peptides
MLRHIQNSLGSVYRSNTATPQGQIIHHRNF

SEQ ID NO: 12 - protein, 35 aa, synthetic construct
  REGION 1..35: note = signal peptides
MNLSLSDLHRQVSRLVQQESGDCTGKLRGNVAANK

SEQ ID NO: 13 - protein, 23 aa, synthetic construct
  REGION 1..23: note = signal peptides
MKKNIAFLLASMFVFSIATNAYA

SEQ ID NO: 14 - protein, 32 aa, synthetic construct
  REGION 1..32: note = signal peptides
MTHKQSGFTLLEVMLAIAILASLSLLAVTVFS

SEQ ID NO: 15 - protein, 23 aa, synthetic construct
  REGION 1..23: note = signal peptides
MQNERGSALLVTLLMLALMAALA

SEQ ID NO: 16 - protein, 25 aa, synthetic construct
  REGION 1..25: note = signal peptides
MLTRFRLTGISLALLLVTSGTPALS

SEQ ID NO: 17 - protein, 23 aa, synthetic construct
  REGION 1..23: note = signal peptides
MTPVAAALLAIAVLAAPPPPRAR

SEQ ID NO: 18 - protein, 30 aa, synthetic construct
  REGION 1..30: note = signal peptides
METSRLLLMLSVAFLALAALIGAILIAYAH

SEQ ID NO: 19 - protein, 15 aa, synthetic construct
  REGION 1..15: note = signal peptides
MRTLTLNELDSVSGG

(19) United States
(12) Patent Application Publication
(10) Pub. No.: US 2025/0177497 A1
(43) Pub. Date: Jun. 5, 2025

(54) NOVEL BACTERIA-BASED DELIVERY SYSTEM FOR TACE (ADAM17) SELECTIVE BIOLOGICAL INHIBITOR

(71) Applicant: Yeda Research and Development Co. Ltd., Rehovot (IL)

(72) Inventors: Irit SAGI, Rehovot (IL); Ravid STRAUSSMAN, Rehovot (IL); Oded SANDLER, Rehovot (IL); Sapir SHCOORY, Rehovot (IL); Inna SOLOMONOV, Rehovot (IL)

(73) Assignee: Yeda Research and Development Co. Ltd., Rehovot (IL)

(21) Appl. No.: 19/054,963

(22) Filed: Feb. 17, 2025

Related U.S. Application Data
(63) Continuation of application No. PCT/IL2023/050877, filed on Aug. 17, 2023.
(60) Provisional application No. 63/398,573, filed on Aug. 17, 2022.

(51) Int. Cl. A61K 38/48; A61K 35/74; A61P 35/00; C12N 1/36; C12N 9/50

(52) U.S. Cl. CPC A61K 38/4886; A61K 35/74; A61P 35/00; C12N 1/36; C12N 9/50

(57) ABSTRACT

Provided are compositions-of-matter comprising non-pathogenic bacteria capable of homing to a tumor, said bacteria comprising a heterologous polynucleotide comprising a nucleic acid sequence encoding a Pro Domain (TPD) polypeptide of TNFα Converting Enzyme (TACE), said TPD being devoid of a catalytic domain of said TACE and said TPD being secreted from or presented on a membrane of said bacteria. Also provided pharmaceutical compositions comprising same and methods of using same for treating cancer.

### RELATED APPLICATIONS

This application is a Continuation of PCT Patent Application No. PCT/IL2023/050877 having International filing date Aug. 17, 2023, which claims the benefit of priority under 35 USC § 119(e) of U.S. Provisional Patent Application No. 63/398,573 filed on Aug. 17, 2022. The contents of the above applications are all incorporated by reference as if fully set forth herein in their entirety.

### SEQUENCE LISTING STATEMENT

The XML file, entitled 102527SequenceListing.xml, created on Jan. 13, 2025, comprising 32,264 bytes, submitted concurrently with the filing of this application is incorporated herein by reference.

### FIELD AND BACKGROUND OF THE INVENTION

The present invention, in some embodiments thereof, relates to compositions-of-matter which secrete or present a TACE pro-domain and, more particularly, but not exclusively, to methods of using same for treating cancer.

Solid malignancies are the most common cancer disorders that cause approximately 93.8% of cancer death cases. Recently, several reports highlighted the importance of the tumor microenvironment (TME) in cancer progression and related death (Peng, D. H. et al. 2020, Nat. Commun. 11:4520); Pickup, M. W., et al. 2014, EMBO Rep. 15:1243-1253). The ECM, a dominant constituent of the TME, harbors remodeling enzymes, signaling molecules, cytokines, and growth factors and serves as a scaffold for migrating cells. Moreover, the TME promotes cancer cell invasion and metastasis creation, leading to tumor recurrence and therapeutic failure. Typically, tumor ECM is over-deposited with collagen, leading to fibrotic environments, which suppress immune cell recruitment and drug penetration²⁻⁵.

Several membrane-bound matrix proteins overexpressed on cancer cells were identified as biomarkers and drug targets for potential prognosis and therapy due to their high expression in diseased tissue (mainly cancer and inflammation) and accessibility at the TME. Of them, unbalanced matrix enzymes such as Matrix metalloproteinases (MMPs), A Disintegrin and Metalloproteinases (ADAMs), and lysyl oxidases (LOX-family enzymes), intensively remodel the pathological fibrotic ECM, promoting tumorigenesis and metastasis (Agren, M. S. et al. 2020, International Journal of Molecular Sciences vol. 21:2678); Dufour, A. et al., 2013. Trends in Pharmacological Sciences 34:233-242).

Targeting enzymes involved in extracellular matrix (ECM) remodeling has shown promise as a therapeutic approach for cancer treatment. However, developing highly selective biological inhibitors against matrix metalloproteinases (MMPs) and members of the A Disintegrin And Metalloproteinase (ADAM) family while maintaining their homeostatic activity in healthy tissues remains a challenge due to their homologous catalytic sites, substrate promiscuity, and ubiquitous tissue and cellular expression. While protein-based inhibitors have been developed with some selectivity, their targeted delivery to affected tissues is still a major challenge. Targeting specific proteolytic cascades within certain organs or tissues by modulating enzymatic catalytic activity selectively presents significant challenges and highlights unfulfilled requirements in the field. In particular, targeting the complex network of proteases involved in ECM remodeling, remains a major unresolved challenge.

The TME consists of various cells, including fibroblasts, endothelial cells, immune cells, and extracellular matrix components such as such as collagen, fibronectin, and laminin, that provide structural support for cells and play a crucial role in cell signaling and migration, and targeting it is a promising approach for cancer therapy (Jin, M. Z. et al., 2020. Signal Transduction and Targeted Therapy, 5:166).

TACE, which is also known as ADAM17, associates with poor clinical outcomes when overexpressed during numerous cancer indications, including Non-small-cell lung carcinoma (NSCLC). TACE is generally produced by activated monocytes and macrophages as an inactive precursor in the endoplasmic reticulum (ER) and becomes mature and active by Furin protease, which cleaves the enzyme pro-domain, TPD (˜25 kDa). TACE has a vital role in the immune system and most of the tissue's homeostasis by shedding more than 70-membrane-bound cytokines when engaged. The shedding activity of TACE orchestrates three central signaling pathways, i.e., TNFα, Interleukin-6 receptor (IL-6R), and Epidermal Growth Factor Receptor (EGF-R), accountable for cancer progression and metastasis. For example, IL-6 interaction with TACE sheds soluble IL-6 receptor, which acts on intestinal epithelial cells via IL-6 trans-signaling to induce colon cancer formation (Das, S. et al. PLoS One 7, 2012); and in NSCLC, releasing soluble IL-6 receptor (sIL-6R) promotes the IL-6 trans-signaling that leads to cancer progression by enhancing cancer cell proliferation, metastasis, and chemotherapy resistance⁹⁻¹⁶.

Several inhibitors of TACE have been developed, such as ZLDI-8 (Zhang, Y. et al. 2018, Cell Death Dis 9(7):743) and TAPI-1 (Gennett M. Myhre et. Al. 2004, Am. J. Physiol. Gastrointest. Liver Physiol., 287: G1213-G1219) and for the IL-6 trans signaling-sgp130 (Schumacher, N. et al., 2019, Cancers, 11:1736). However, despite intensive efforts synthetic drugs have failed in many clinical trials due to their lack of specificity, efficacy, and off-target effects.

Recently, the major role of TME-TACE in NSCLC metastasis was demonstrated (Bolik, J. et al. 2021. Journal of Experimental Medicine, vol. 219(1), e20201039). In addition, selective endogenous-like biological inhibitors against several matrix enzymes were generated, including the form of the enzyme pro-domain (Wong, E. et al. 2016, Sci Rep 6, 1-12).

Additional background art includes Massa P. et al. 2013 (Blood, 122(5):705-714); Wen, M. et al. 2018, Cancer Lett. 433, 140-146; WO2013/168164 (Sagi Irit et al., published 14 Nov. 2013); and WO2022/175951 (STRAUSSMAN Ravid et al., published 25 Aug. 2022).

### SUMMARY OF THE INVENTION

According to an aspect of some embodiments of the present invention there is provided a composition-of-matter comprising non-pathogenic bacteria capable of homing to a tumor, the bacteria comprising a heterologous polynucleotide comprising a nucleic acid sequence encoding a Pro Domain (TPD) polypeptide of TNFα Converting Enzyme (TACE), the TPD being devoid of a catalytic domain of the TACE and the TPD being secreted from or presented on a membrane of the bacteria.

According to an aspect of some embodiments of the present invention there is provided a pharmaceutical composition comprising a therapeutically effective amount of the composition-of-matter of some embodiments of the invention, and a therapeutically acceptable carrier.

According to an aspect of some embodiments of the present invention there is provided a method of treating cancer in a subject, comprising administering to the subject a therapeutically effective amount of composition-of-matter of some embodiments of the invention or the pharmaceutical composition of some embodiments of the invention, thereby treating the subject.

According to an aspect of some embodiments of the present invention there is provided a composition-of-matter of some embodiments of the invention or a pharmaceutical composition of some embodiments of the invention, for use in treating cancer.

According to some embodiments of the invention, the non-pathogenic bacteria is an attenuated Salmonella.

According to some embodiments of the invention, the non-pathogenic bacteria is an attenuated pseudomonas aeruginosa (CHA-OST).

According to some embodiments of the invention, the Salmonella is Salmonella Typhimurium strain VNP20009 (STM-YS1646).

According to some embodiments of the invention, the non-pathogenic bacteria comprises modified lipopolysaccharides.

According to some embodiments of the invention, the heterologous polynucleotide further comprises a nucleic acid sequence encoding a secretion signal peptide (SSP) being translationally fused to the nucleic acid sequence encoding the TPD polypeptide.

According to some embodiments of the invention, the heterologous polynucleotide further comprises a nucleic acid sequence for membrane anchorage or presentation of the TPD.

According to some embodiments of the invention, the TPD polypeptide comprises a modification which renders resistant of the TPD polypeptide to furin degradation.

According to some embodiments of the invention, the modification is at a position selected from the group consisting of R⁵⁸, R⁵⁶, K⁵⁷, R²¹¹, R²¹⁴, and C¹⁸⁴.

According to some embodiments of the invention, the nucleic acid sequence encoding the TPD is operably linked to a constitutive promoter.

According to some embodiments of the invention, the nucleic acid sequence encoding the TPD is operably linked to an inducible promoter specifically active under hypoxia.

According to some embodiments of the invention, the non-pathogenic bacterium is capable of specifically proliferating under hypoxia conditions in a tumor microenvironment.

According to some embodiments of the invention, the non-pathogenic bacteria is capable of specifically proliferating under necrosis in a tumor microenvironment.

According to some embodiments of the invention, the composition-of-matter of some embodiments of the invention, further comprising at least one cancer therapeutic.

According to some embodiments of the invention, the cancer comprises a solid tumor.

According to some embodiments of the invention, the cancer comprises a non-solid tumor.

According to some embodiments of the invention, the cancer comprises cancer metastases.

According to some embodiments of the invention, the cancer comprises lung cancer.

According to some embodiments of the invention, the cancer is characterized by abnormal extracellular matrix (ECM) deposition and remodeling.

According to some embodiments of the invention, the therapeutically effective amount of the composition-of-matter is selected capable of decreasing collagen levels in a tumor microenvironment.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIG. 1 is a schematic illustration depicting the TNFα converting enzyme (TACE) as a key regulator of several biological activities. For example, targeting TACE and IL-6 trans-signaling offers a promising therapeutic approach for cancer treatment, such as for cancers involving abnormal ECM deposition and remodeling.

FIG. 2 is a schematic illustration depicting tumor-specific delivery of TPD by attenuated STM.

FIGS. 3A-C demonstrate that TPD decreases the viability of lung cancer cells and inhibits p-ERK. FIG. 3A—LLC cells were treated with TPD (0-16 μM) for 16 hours, and cell viability was monitored using the luminescent cell viability assay. N=10, p=0.0168, R²=0.642, IC₅₀=16.697. FIG. 3B—Representative Western-Blot of TACE and p-ERK of LLC1 under TPD treatment. Tubulin was used as an internal control. FIG. 3C—The relative level of p-ERK protein levels in TPD-treated LLC1 cells was determined by densitometry. N=3.

FIGS. 4A-D depict that TPD treatment moderately improves the survival of mice with KRAS-mutant non-small cell lung cancer (NSCLC). FIG. 4A—A chronological scheme illustrates the syngeneic lung cancer model used in the study. FIG. 4B—Tumor volume measurements were taken for mice with LLC1 KRAS-mutant lung cancer starting from day −7. FIG. 4C—Weight measurements were taken for mice with LLC1 tumors under TPD treatment. The data are presented as means. FIG. 4D—The Kaplan-Meier curves show the survival of mice with different treatments, monitored for 22 days after tumor implantation. The sample size was N=5.

FIGS. 5A-E depict a genetic modification of an attenuated Salmonella STM-YS1646 (STM) strain to over-secret TPD. FIG. 5A—Schematic representation of sspH1-TPD. FIG. 5B—Prediction of ssPH1-TPD structure using AlphaFold deep learning algorithm. FIGS. 5C-D—Four different STM-TPD colonies were grown overnight in LB medium in order to express and validate sspH1-TPD. The bacterial culture was split into supernatant and pellet fractions by centrifuge and was analyzed by Western Blot using an antibody against his-tag. FIG. 5C—Western Blot of STM-TPD and STM conditioned media. FIG. 5D—Western Blot of STM-TPD bacterial lysate. FIG. 5E—The STM secreted-TPD levels were determined by densitometry analysis of pure TPD known amounts.

FIGS. 6A-D demonstrate that the engineered STM-TPD conditioned media modulate TNFα secretion and inhibit lung cancer cell viability in cell-based assay. FIG. 6A—TACE inhibition by STM-TPD secretion was estimated by measuring TACE substrate, TNFα. Macrophages J7 cells were treated for 3 hours with increasing concentrations (0-16 μM) of a conditioned media of STM-TPD (shown by pink triangles), STM-OVA (shown by green circles), and STM control (shown by black squares) and TNFα levels were monitored by ELISA. N=3. IC₅₀ of STM-TPD=8.8 μM±18.069. FIG. 6B—LLC1 cells were treated for 16 hours with a conditioned media of STM-TPD (shown by pink triangles), STM-OVA (shown by green circles), and STM control (shown by black squares) at a concentration from 0-75%, and cell viability was monitored using luminescence cells viability assay. N=10, p<0.001. FIGS. 6C-D are schemes depicting a viability assay protocol (FIG. 6C) and a TNFα secretion assay protocol (FIG. 6D) according to some embodiments of the invention.

FIGS. 7A-I depict the effectiveness of STM-TPD treatment in LLC1-s.c tumors with minimal off-target to non-tumorigenic tissues. FIG. 7A—The diagram depicts the in-vivo bacteria treatment of LLC1 syngeneic lung cancer mice. FIG. 7B—STM-TPD selectively accumulates in the TME in mice. Mice with S.C LLC1 tumors were given intravenous doses of 1×10⁵ and 1×10⁶ CFU of STM-TPD once, and the tumors were collected after ten days after bacteria injections. CFU counting on ampicillin LB plates determined the amount of accumulated STM-TPD in tumors, livers, and spleens on the 10th day after infection. FIG. 7C—Bacterial-tumor specificity was determined by CFU quantification using visual scoring and open CFU. The mean±SEM values of four mice are shown. FIG. 7D—depicts STM-TPD bacteria migrated specifically into the LLC1-s.c tumors. Preferential accumulation of Salmonella (S.C.) in the tumor microenvironment of mice administered intravenously (i.v) with STM-TPD. Mice bearing LLC1 tumors were i.v administered with 1×10⁵ and 1×10⁶ CFU of STM-TPD and following 10 days the mice were sacrificed. The amounts of accumulated STM-TPD in tumors, livers, and spleens were determined on 10-day post infection (p.i.) by CFU counting on ampicillin LB plates as shown in FIG. 7B. Each value represents mean±SEM from 4 mice; FIGS. 7E-I—in vivo toxicity of engineered Salmonella expressing TPD. LLC1 tumor-bearing mice were divided by shuffling into groups of five mice each and i.v injected with 100 μl of PBS or 1, 3, 6×10⁶ CFU of attenuated Salmonella YS1646 (STM) carrying pQE60 plasmid expressing TPD. FIG. 7E—Body weight of tumor-bearing mice was recorded every 2-3 days. FIG. 7F—Tumor volume of mice from different groups were measured. FIGS. 7G-H—Body weight (FIG. 7G) and tumor growth rate (FIG. 7H) of each mouse is shown separately. FIG. 7I—Survival proportions of the groups. Symbols: Black circles=Control PBS; pink squares=1×10⁶ CFU STM-TPD; green triangles=3×10⁶ CFU STM-TPD; purple triangles=6×10⁶ CFU STM-TPD. It is noted that at a dose of 6×10⁶ CFU STM-TPD the mice died 1 day post injection, as can be seen in FIG. 7I (the survival graph), where there is only one measure of that group on day 0.

FIGS. 8A-G depict the efficacy of STM-TPD therapeutic administration in eliminating macro-metastasis in KRAS-mutant lung cancer. FIG. 8A—A chronological scheme that describes the in-vivo metastasis model used in the study. FIG. 8B—Tissue metastases of LLC1 were evaluated 3.5 weeks after bacteria injection and treated with different agents, including STM-TPD, PBS, recombinant TPD, STM-Ova, and unmodified-STM. FIG. 8C—CFU counting on ampicillin/chloramphenicol antibiotics L.B. plates 3.5 weeks post-bacteria injection (I.V.) was used to determine the amounts of accumulated bacteria in the livers and spleens of mice (N=3). FIG. 8D—Lung metastases of LLC1 were assessed 3.5 weeks post-bacteria injection and treated with different agents, including STM-TPD, PBS, STM-Ova, and unmodified-STM. The results showed a significant decrease in lung metastasis after STM-TPD treatment. FIGS. 8E-G—The effectiveness of the treatment was determined by visual scoring (FIG. 8E) and by new software for quantifying organ metastasis (FIGS. 8F-G).

FIGS. 9A-B depict the inhibitory effect of STM-TPD In-vivo. FIG. 9A—Tumor volume measurements of mice with LLC1 KRAS-mutant lung cancer implanted with STM-TPD and TPD 4 mg/kg, PBS, STM-Ova, and unmodified STM on 100 mm³ for 20 days after treatment implantation. STM-TPD attenuates tumor growth In-vivo, demonstrating a stronger and improved biological impact. N=4. FIG. 9B—Weight measurements of mice. N=4.

FIGS. 10A-G demonstrate that STM-TPD treatment decreases ECM proteins in LLC tumors. FIGS. 10A-B—S.C-LLC tumors collected 14 days post-treatment (STM-TPD, STM-Ova, STM, TPD, and PBS). FIG. 10A—H&E. MTC and SR sections showed decreased collagen deposition. FIG. 10B—Quantification of SR staining shows a significant decrease in collagen deposition, n=3. FIGS. 10C-D—Degradomics analysis of TPD-treated LLC S.C tumors shows a significant decrease in ECM proteins. 7000 N-terminal peptides were quantified, of which 220 were differentially expressed between the treatment groups. FIG. 10C—Network visualization and pathway analysis showed enrichment of Cell junction proteins and TNF-alpha signaling. FIG. 10D—Hierarchical cluster analysis revealing degradome and heatmap with distinct protein expression profiles in LLC-S.C tumors. Downregulated and upregulated genes are shown in green and red blocks, respectively. Specific proteins are highlighted here. Several ECM substrates are Collagen 5A3, NOTCH2, Serpin A1D, MMP-3, Fibronectin, Laminin beta-2, Peroxidasin, Thrombospondin-3, Semaphorin-6D. FIG. 10E—Western blot of fibronectin and MMP13. Actin was used as a loading control. FIGS. 10F-G—The relative levels of fibronectin and MMP13 protein levels were determined by densitometry.

FIGS. 11A-K depict that STM-TPD secretes TPD, decreasing IL-6 trans-signaling, cyclin-D1 (CDK1), and TNFα levels in LLC tumors. FIG. 11A—a schematic demonstrating that LLC1 subcutaneous tumors were harvested 14 days after bacteria injection into tumor-bearing mice. FIGS. 11B-C—Blood serum ELISA 48 hours after bacteria administration for IL-6 (FIG. 11B) and sIL-6R (FIG. 11C). FIGS. 11D-F depict Western blot analyses for ERK, α-SMA, CDK1 (FIG. 11D), p-ERK (FIG. 11E), and HIS (FIG. 11F). Tubulin (a housekeeping protein) was used an internal control for amounts of proteins. FIG. 11G—a graph depicting quantification of ERK activation in the indicated treatments. FIG. 11H—a graph depicting Cyclin D1 levels in the indicated treatments. FIG. 11I—a graph ELISA of blood serum sTNFα 48 hours post bacteria injection. FIGS. 11J-K—The relative levels of αSMA (FIG. 11J) and HIS (FIG. 11K) protein levels were determined by densitometry.

FIGS. 12A-E demonstrate that TPD shows an antitumor effect while a PD1 did not affect growth rate in KRAS lung cancer. FIG. 12A—A schematic representation of TPD. FIG. 12B—Tumor volume measurements of mice with LLC1 KRAS-mutant lung cancer implanted on day −7. Data are represented as means±SEM. Statistical significance was determined with two-way analysis of variance (ANOVA) and Tukey's multiple comparisons tests. FIG. 12C—Kaplan-Meier curves comparing combinations of TPD, a-PD1, and untreated control mice monitoring survival for 22 days after tumor implantation. N=5. FIG. 12D—Weight measurements of mice with LLC1 tumors under TPD and a-PD1 treatment. Data are represented as means and SEM. FIG. 12E—Tumor volume and survival over time per mouse.

FIG. 13 Schematic illustration depicting some embodiments of the invention employing the STM-TPD in in-vivo and in-vitro experimental designs.

FIG. 14 depicts a schematic illustrations of experimental designs according to some embodiments of the invention. “ICV”=inhibitory cellular vehicles; “FME”=fibrotic microenvironments; “ERD”=ECM-related diseases.

FIGS. 15A-C depict an in-vivo syngeneic lung cancer model calibration. FIG. 15A—A chronological scheme describes the in-vivo syngeneic lung cancer model. FIG. 15B—Average tumor volume measurement in three treatment groups: 0.25×10⁶ cells, 1×10⁶ cells, and 3×10⁶ cells. Measurements were taken on days 5, 7, 9, 12, 14 post-injections. Data are represented as means±SEM, N=4. FIG. 15C—Average body weight; data is represented as means±SEM, N=4.

FIGS. 16A-B depict that TPD delivery improves its anti-tumor effect. FIG. 16A—Weight measurements of mice with LLC1 tumors under STM-TPD and TPD 4 mg/kg. PBS, STM and unmodified STM were used as a controls. FIG. 16B—Tumor volume measurements of mice with LLC1 KRAS-mutant lung cancer implanted with STM-TPD and TPD 4 mg/kg. PBS, STM and unmodified STM on 100 mm³ for 20 days after treatments implantation. N=4.

FIG. 17 depicts a schematic illustration of a construct designed for presentation of the TPD on the surface of the non-pathogenic bacteria of some embodiments of the invention.

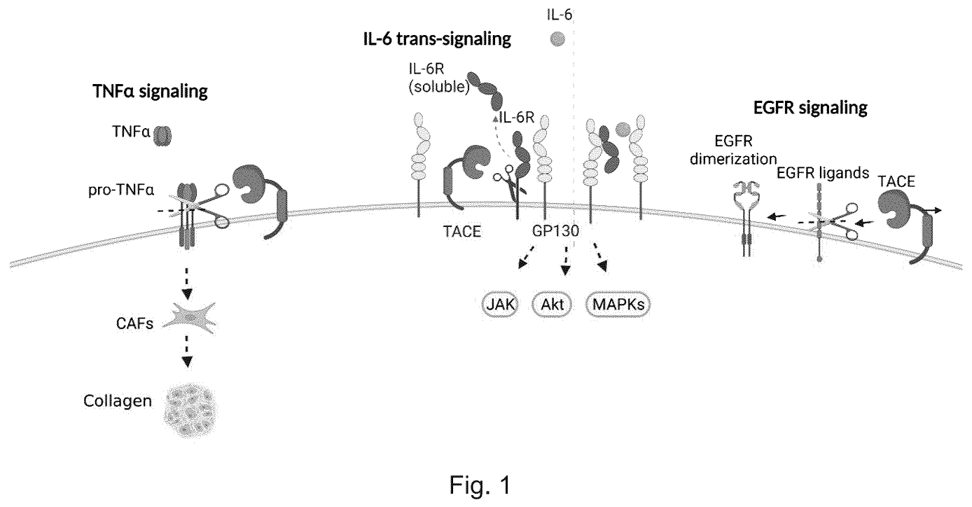

### DESCRIPTION OF SPECIFIC EMBODIMENTS OF THE INVENTION

The present invention, in some embodiments thereof, relates to compositions-of-matter which secrete or present a TACE pro-domain and, more particularly, but not exclusively, to methods of using same for treating cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

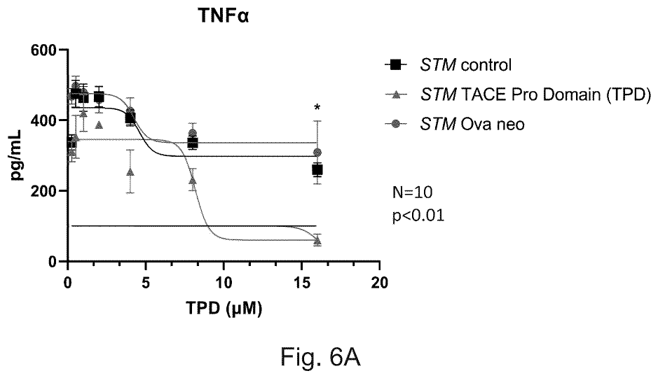

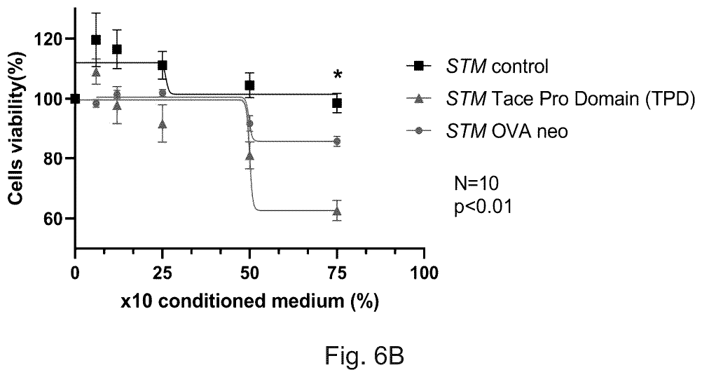

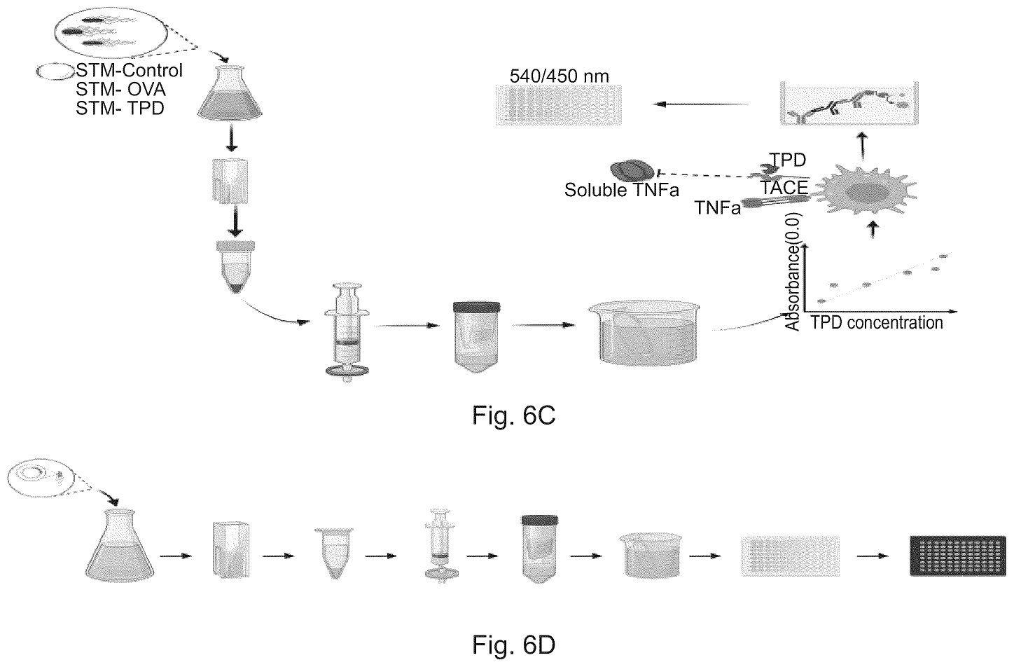

The present inventors have engineered novel bacterial-based delivery systems designed to secrete TPD to the TME. The STM-TPD was tested in-vitro and in-vivo (e.g., shown in FIGS. 6A-D, 7A-I, 8A-G, 9A-B, 10A-G, 11A-K, 12A-E and 13). Examples 4 and 9 of the Examples section which follows show that secreted TPD from STM-TPD inhibited the shedding of pro-TNFα from the cell surface and decreased cancer cell viability. Remarkably, in-vivo, treatment of STM-TPD particles greatly improved homing of TPD in the TME and significantly reduced metastasis and tumor progression over treatment with TPD which is not delivered by the STM-TPD. In addition, STM-TPD reduced the TNFα and IL-6 trans-signaling in blood serum and reduces the collagen deposition and ERK signaling within the TME. Overall, STM-TPD provides a general yet novel drug delivery system to administer anti-matrix biological inhibitors, e.g., TPD, directly into the TME.

According to an aspect of some embodiments of the invention there is provided a composition-of-matter comprising non-pathogenic bacteria capable of homing to a tumor, the bacteria comprising a heterologous polynucleotide comprising a nucleic acid sequence encoding a Pro Domain (TPD) polypeptide of TNFα Converting Enzyme (TACE), the TPD being devoid of a catalytic domain of the TACE and the TPD being secreted from or presented on a membrane of the bacteria.

As used herein the phrase “non-pathogenic bacteria” refers to bacteria which are not capable of causing disease.

Bacterial infection often overstimulate induction of TNFα, which leads to a cytokine cascade which may result in a sepsis shock in human. In gram-negative bacteria such effect is due to the lipopolysaccharide moiety on the bacterial cells (Clairmont C, et al., 2000. “Biodistribution and Genetic Stability of the Novel Antitumor Agent VNP20009, a Genetically Modified Strain of Salmonella typhimurium”, The Journal of Infectious Diseases 2000; 181:1996-2002).

According to some embodiments of the invention, the non-pathogenic bacteria does not induce TNFα overstimulation which can cause sepsis in a subject (e.g., a human subject).

Various tools have been shown to prevent the induction of TNFα overstimulation by bacteria, including disruption of the msbB and purl genes. The msbB gene is responsible for myristoylation of lipid A, the hydrophobic group of lipopolysaccharide, which covers the surface of most Gram-negative bacteria. Deletion of msbB reduced Salmonella's toxicity by 10,000-fold while retaining its anti-tumor efficacy in mice (reviewed in Clairmont C., et al., 2000 (Supra).

According to some embodiments of the invention, the non-pathogenic bacteria is devoid of lipopolysaccharides (LPS) or has a modified LPS.

According to some embodiments of the invention, the non-pathogenic bacteria has a chromosomal deletion of purl and msbB.

According to some embodiments of the invention, the non-pathogenic bacteria is Purin auxotrophic.

According to some embodiments of the invention, the non-pathogenic bacteria is an attenuated Salmonella.

According to some embodiments of the invention, the attenuated Salmonella is Salmonella Typhimurium strain VNP20009 (STM-YS1646).

According to some embodiments of the invention, the attenuated Salmonella is Typhimurium strain STM3210.

According to some embodiments of the invention, the non-pathogenic bacteria is an attenuated pseudomonas aeruginosa (CHA-OST).

According to some embodiments of the invention, the non-pathogenic bacteria is selected from the group consisting of STM VPN20009, STM Ty21a, STM yB1, E.Coli mg1655, E.Coli Nissle 1917, proteus, Lactobacillus, E.coli SYNB1891, Brucella Calmette Guerin, and Listeria monocytogenes.

The phrase “homing to a tumor” as used herein refers to the preferential accumulation of bacterial cells at a tumor site which depends on conditions such as pH (e.g., acidity), oxygen level (e.g., hypoxia), necrosis, and/or tumor-associated metabolite(s) present in the tumor and/or the tumor microenvironment.

According to some embodiments of the invention, the tumor is a solid tumor.

According to some embodiments of the invention, the tumor is a non-solid tumor.

The term “tumor microenvironment” or “TME” refers to the area surrounding the solid tumor.

The cell population at the TME is rather heterogenic. Thus it is composed from cancer cells, somatic cells, immune cells and stem cells. These cells are all prone to dynamic changes in their genetic sub-typing that is often directly influenced by the environmental cues such as matrix deposition, remodeling metabolic shifts and stem cell aberrant differentiation.

Tumor microenvironment typically comprises blood vessels, fibroblasts, endothelial cells, immune cells, signaling molecules, and the extracellular matrix.

The extracellular matrix (ECM) typically contains collagen, fibronectin, and laminin.

According to some embodiments of the invention, the TME is characterized by hypoxia conditions.

According to some embodiments of the invention, the hypoxia conditions in the TME comprise an oxygen level in the form of O2 which is less than 4%, e.g., less than 3%, less than 2.5%, less than 2%, less than 1.5%, e.g., less than 1%.

According to some embodiments of the invention, the TME is characterized by necrosis. The term “necrosis” as used herein refers to the death of cells in the TME.

According to some embodiments of the invention, proliferation of the non-pathogenic bacteria is preferred under hypoxia conditions (e.g., such as those present in the TME) as compared to under normoxia conditions (e.g., such as the normal oxygen levels present in healthy (non-cancerous) tissues in the human body).

According to some embodiments of the invention, the non-pathogenic bacteria is capable of specifically proliferating under hypoxia conditions in the tumor microenvironment.

According to some embodiments of the invention, the non-pathogenic bacterium is capable of specifically proliferating under necrosis in a tumor microenvironment.

Some non-pathogenic bacteria such as Salmonella can nourish from tumor metabolites post infection. For example, the Salmonella can nourish from glucose which is accumulated near the cancerous cells, for example, in the TME.

According to some embodiments of the invention, proliferation of the non-pathogenic bacteria is preferred in the presence of tumor metabolites.

According to some embodiments of the invention, the non-pathogenic bacteria resides at the tumor site, e.g., in the TME, but not in health tissues of a subject.

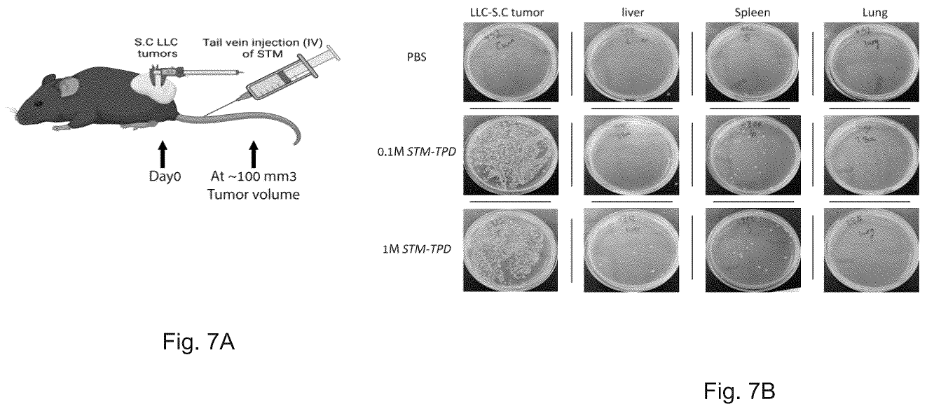

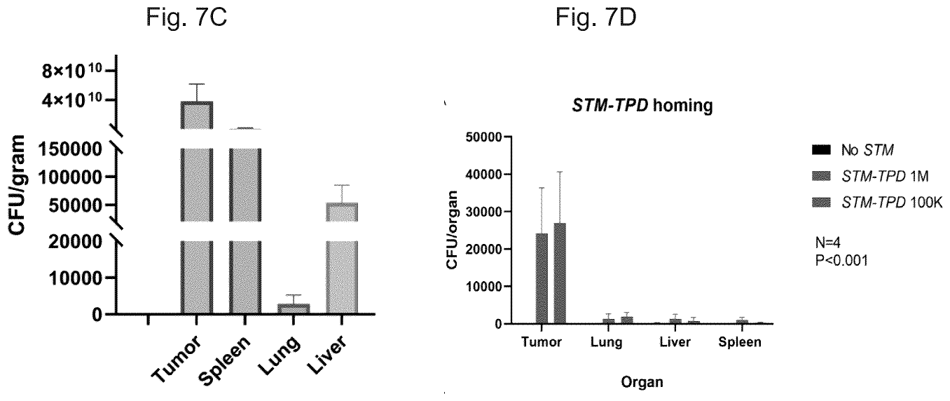

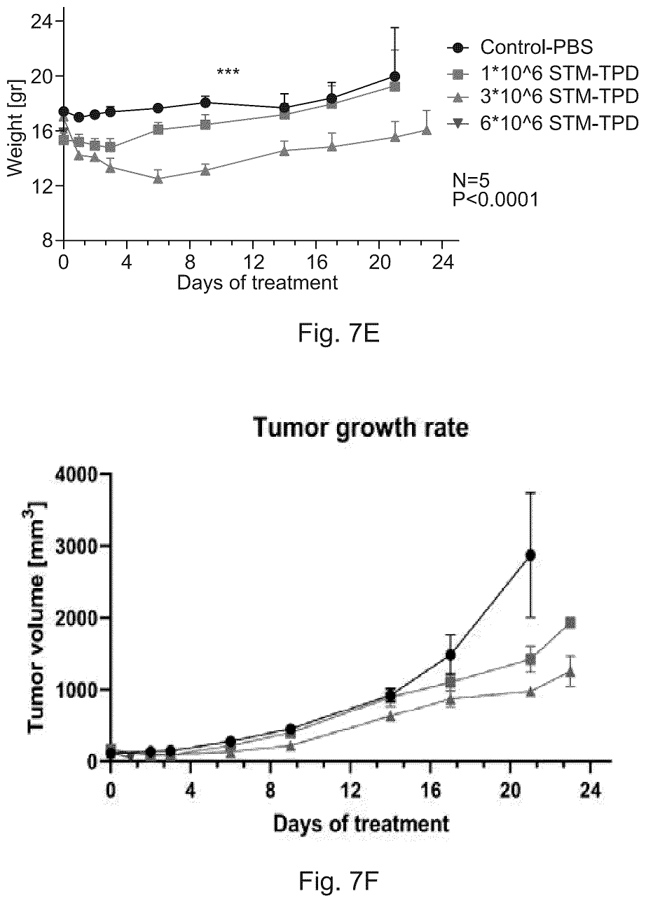

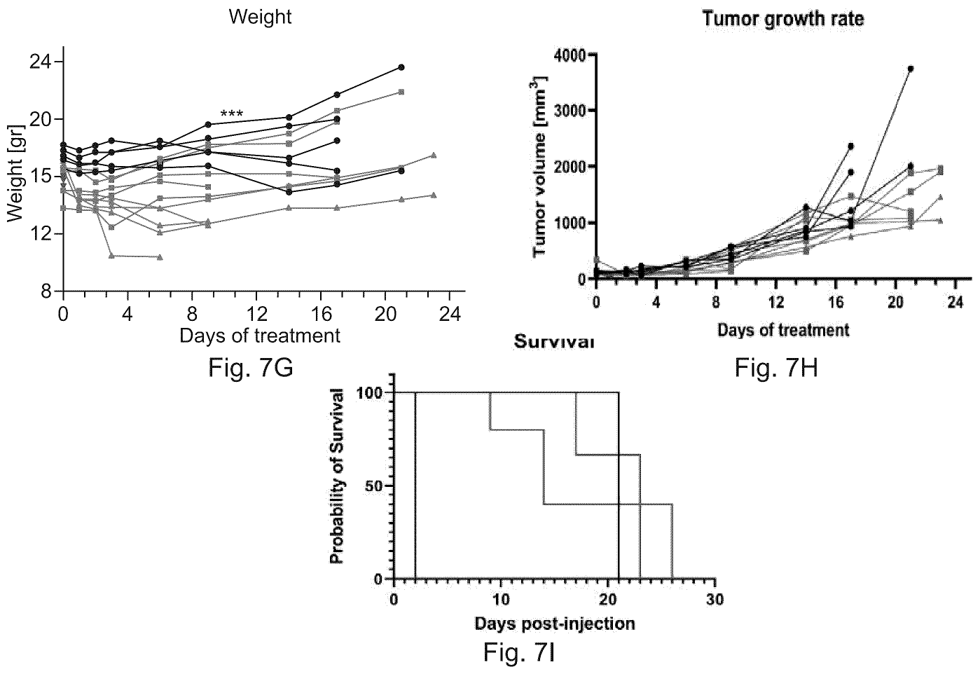

For example, as shown in FIGS. 7B and 7D, and as described in Example 5 of the Examples section which follows, the bacteria comprising the TPD was shown capable of specifically proliferating in the tumor site and not in healthy tissues such as liver, spleen and lung.

TACE (tumor necrosis factor-α-converting enzyme; EC number 3.4.24.86), also called ADAM metallopeptidase domain 17 (ADAM17), is a 70-kDa enzyme that belongs to the ADAM protein family of disintegrins and metalloproteases.

Similar to other members of the Matrix metalloprotease (MMP) family, TACE is generated as a latent zymogen and is activated upon the release of the inhibitory pro-domain. The activation of TACE zymogen is performed mainly by a Furin-like protease, a proprotein convertase, in the late Golgi compartment.

As used herein, the phrase “pro-domain of TNF-α converting enzyme (TACE)” refers to the polypeptide portion of TACE that is responsible for maintaining the enzyme in its inactive form (i.e. not comprising catalytic activity).

According to one embodiment, the mutated pro-domain is derived from a human TACE, although other mammalian sequences of TACE are also contemplated.

The mRNA and amino acid sequences for Homo sapiens TACE can be found under GenBank Accession No. NM_003183 (SEQ ID NO:5) encoding the amino acid sequence set forth by GenBank Accession No. NP_003174.3 (SEQ ID NO:6).

According to a particular embodiment, the TACE pro-domain comprises the polypeptide sequence from Asp²³-Arg²¹⁴ of full length TACE.

According to some embodiments of the invention, the TPD comprises the native (wild-type) amino acid sequence without any modification with respect to the TPD sequence of SEQ ID NO:2.

For example, SEQ ID NO:2 depicts the amino acid sequence which was expressed in and secreted by the attenuated Salmonella (STM-TPD) used in the in-vitro and in-vivo experiments described in the Examples section which follows.

According to some embodiments of the invention, the TPD polypeptide comprises a modification which renders resistant of the TPD polypeptide to furin degradation.

The phrase “resistant to furin degradation” as used herein refers to having a higher resistance such as at least 10% more resistant, at least 20% more resistant, at least 30% more resistant, at least 40% more resistant, at least 50% more resistant to furin degradation than the native sequence under the same reaction conditions. Analyzing the furin resistance of the polypeptide may be effected by incubating the polypeptide in the presence of furin and analyzing for the generation of fragments (e.g. by SDS gel analysis).

According to some embodiments of the invention, the modification is at a position selected from the group consisting of R⁵⁸, R⁵⁶, K⁵⁷, R²¹¹, R²¹⁴, and C¹⁸⁴.

It will be appreciated that the numbering of the mutations corresponds to the full length TACE enzyme and not the TACE pro-domain.

According to some embodiments of the invention, the R⁵⁸, R⁵⁶, K⁵⁷, R²¹¹, or R²¹⁴ is replaced by Alanine, Asparagine, Aspartic Acid, Cysteine, Glutamine, Glutamic Acid, Glycine, Histidine, Isoleucine, Leucine, Methionine, Phenylalanine, Proline, Serine, Threonine, Tryptophan, Tyrosine or Valine.

According to some embodiments of the invention, the C¹⁸⁴ is replaced by alanine, or valine.

According to some embodiments of the invention, the C¹⁸⁴ is replaced by alanine.

According to some embodiments of the invention, the R⁵⁸ is replaced by alanine.

According to some embodiments of the invention, the R²¹¹ is replaced by alanine.

According to some embodiments of the invention, the R²¹⁴ is replaced by glycine.

According to some embodiments of the invention, the replacing amino acids are not positively charged amino acids—e.g. arginine or lysine.

As mentioned, the pro-domain of this aspect of the present invention is devoid of TACE catalytic (sheddase) activity.

As mentioned, the TPD is being secreted from or presented on a membrane of the bacteria.

As used herein the term “secreted” refers to a polypeptide which is secreted from membrane of the bacteria and hence is soluble.

According to some embodiments of the invention, the TPD polypeptide is secreted from the non-pathogenic bacteria into the tumor microenvironment.

As used herein the term “presented on a membrane of the bacteria” refers to a polypeptide which, while being still attached to the bacterial membrane is exposed to the microenvironment of the bacteria, e.g., to the outer surface of the bacteria.

It should be noted that a polypeptide which is presented on the membrane of the bacteria can form part of a transmembrane polypeptide of the bacteria.

For example, the coding sequence of the TPD polypeptide can be translationally fused to a coding sequence of a membrane anchored polypeptide, e.g., a polypeptide which is presented on the surface (i.e., outer surface) of the bacterial cell.

The phrase “being translationally fused” as used herein refers to being encoded by a single open reading frame (ORF).

According to some embodiments of the invention, the TPD polypeptide is embedded within the sequence of the membrane polypeptide presented on a surface of the non-pathogenic bacteria. For example, in this embodiment, the nucleic acid sequence encoding the TPD is embedded within a nucleic acid sequence encoding a membrane polypeptide presented on a surface of the non-pathogenic bacteria.

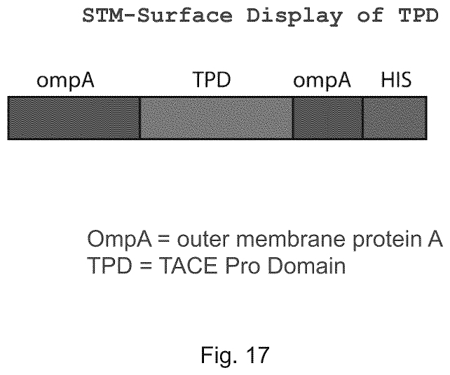

For example, FIG. 17 schematically depicts an embodiment of the invention in which the coding sequence of TPD is embedded within the coding sequence of outer membrane protein A (OmpA).

Additionally or alternatively, a polypeptide (e.g., the TPD polypeptide) can be attached by chemical attachment to the surface of a bacteria. Such attachment can be any suitable chemical linkage, direct or indirect, as via a peptide bond, or via covalent bonding to an intervening linker element, such as a linker peptide or other chemical moiety, such as an organic polymer. The polypeptide may be linked via bonding at the carboxy (C) or amino (N) termini, or via bonding to internal chemical groups such as straight, branched or cyclic side chains, internal carbon or nitrogen atoms, and the like.

In some embodiments, therapeutic agents (e.g., the TPD polypeptide and/or the at least one cancer therapeutic described hereinunder) are attached to the outside of the bacteria using an attachment method such as CLICK chemistry. Such methods are further described in U.S. Patent Application No. 20200087703 and U.S. Patent Application No. 20200054739, the contents of which are incorporated herein by reference.

According to some embodiments of the invention, the TPD polypeptide is attached by chemical attachment to the surface of the non-pathogenic bacteria.

As used herein the term “heterologous” refers to a nucleic acid sequence which may not be naturally expressed by the non-pathogenic bacteria.

The heterologous polynucleotide can be inserted into the non-pathogenic bacteria as part of a nucleic acid construct suitable for expression in bacteria. The heterologous polynucleotide or the construct can be integrated into the genome of the bacteria (e.g., as described in Canale F. P. et al., 2021; Nature 598, 662-666; which is fully incorporated herein by reference) or remain as a plasmid.

For example, in some embodiments, the nucleic acid encoding the TPD polypeptide is operably linked to transcriptional regulatory elements, such as a bacterial promoter.

Examples of bacterial promoters include but are not limited to STM1787 promoter, pepT promoter, pflE promoter, ansB promoter, vhb promoter, FF+20* promoter or p(luxI) promoter.

In some embodiments, the polypeptide (e.g., the TPD polypeptide) is constitutively expressed by the bacteria.

Non-limiting examples of constitutive promoters which can be used by the heterologous polynucleotide of some embodiments of the invention, include, but are not limited to PagC, stnYp and spiC.

In some embodiments, the TPD polypeptide is expressed by the bacteria in an inducible manner (e.g., it is expressed upon exposure to conditions present in the tumor microenvironment). Such conditions include for example, acidity and hypoxia.

For example, the STM1787 promoter is sensitive to the acidic microenvironment of tumors [Fliente K, et al. 2012. “A bioluminescent transposon reporter-trap identifies tumor-specific microenvironment-induced promoters in Salmonella for conditional bacterial-based tumor therapy”. Cancer Discov. 2:624-37]. According to some embodiments of the invention, the nucleic acid sequence encoding the TPD is operably linked to an inducible promoter specifically active under hypoxia.

Non-limiting examples of inducible promoters specifically active under hypoxia which can be used by the heterologous polynucleotide of some embodiments of the invention, include, but are not limited to pflE and ansB (described in Nabil Arrach et al., 2008 (“Salmonella Promoters Preferentially Activated Inside Tumors”; Cancer Res. 68(12):4827-4832), mltD, glpA, and glpT (described in Sara Leschner et al., 2012, Nucleic Acids Research, 40(7):2984-2994) and adhE (described in Jianxiang Chen et al. 2011. Molecular & Cellular Proteomics 10:1-11).

The transcriptional regulatory element can further comprise a secretion signal. The secretion signal peptide enables the secretion of the TDP polypeptide from the non-pathogenic bacteria.

Bacterial secretion systems are well known in the art (e.g., reviewed in Erin R. Green and Joan Mecsas, 2016. “Bacterial Secretion Systems—An overview”, Microbiol Spectr. 4(1):10.1128/microbiolspec. VMBF-0012-2015; which is fully incorporated herein by reference). Non-limiting examples of bacterial secretion system include, the Type III Secretion System (T3SS), T4SS, T5SS, T6SS, and T7SS.

The bacterial secretion apparatus may utilize a secretion signal which is present at the N-terminus or the C-terminus of the secreted polypeptide. Examples of bacterial secretion apparatuses include, but are not limited to, Sec (exists in both Gram (+) and Gram (−) bacteria; secretion signal is at N-terminus); Tat (exists in both Gram (+) and Gram (−) bacteria; secretion signal is at N-terminus); T1SS (exists in Gram (−) bacteria; secretion signal is at C-terminus); T2SS (exists in Gram (−) bacteria; secretion signal is at N-terminus); T3SS (exists in Gram (−) bacteria; secretion signal is at N-terminus); T4SS (exists in Gram (−) bacteria; secretion signal is at C-terminus); T5SS (exists in Gram (−) bacteria; secretion signal is at N-terminus); SecA2 (exists in Gram (+) bacteria; secretion signal is at N-terminus); Sortase (exists in Gram (+) bacteria; secretion signal is at N-terminus); Injectosome (exists in Gram (+) bacteria; secretion signal is at N-terminus); and T7SS (exists in Gram (+) bacteria; secretion signal is at C-terminus).

According to some embodiments of the invention, the heterologous polynucleotide further comprises a nucleic acid sequence encoding a secretion signal peptide (SSP) being translationally fused to the nucleic acid sequence encoding the TPD polypeptide.

According to some embodiments of the invention, the coding sequence of the secretion signal peptide is placed upstream of the coding sequence encoding the TPD polypeptide, hence the secretion signal peptide is placed at the N-terminus of the TPD polypeptide.

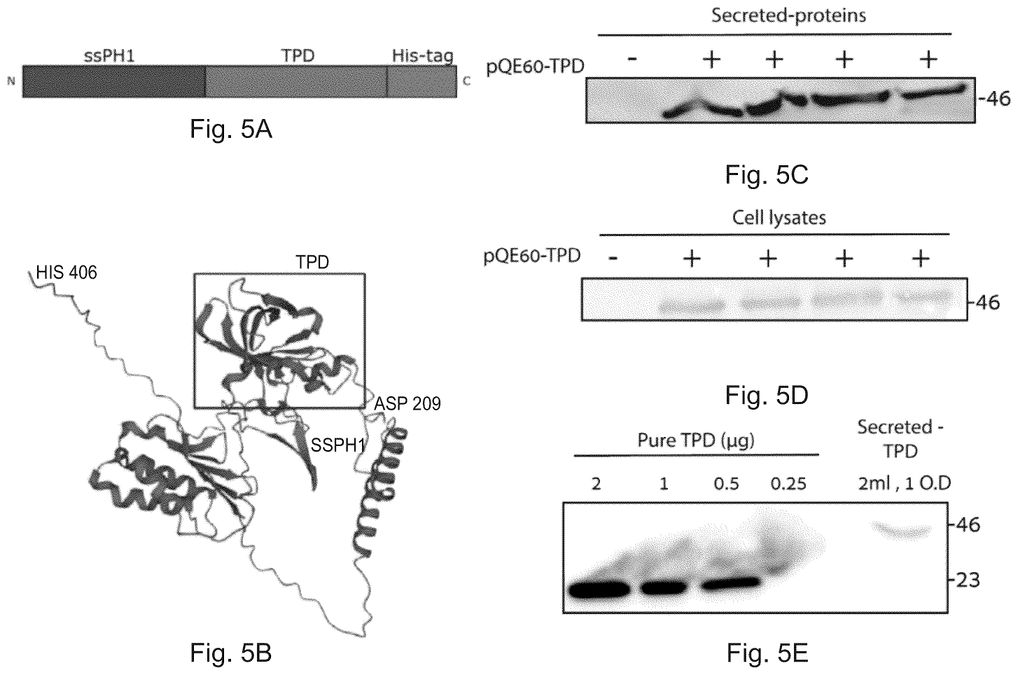

For example, FIG. 5A schematically depicts a heterologous polynucleotide according to some embodiments of the invention in which the secretion signal peptide is upstream of the TPD coding sequence.

For example, the present inventors have used the SSP sequence set forth by SEQ ID NO:7, which is encoded by the SSPH1 coding sequence set forth by SEQ ID NO:3.

According to some embodiments of the invention, the coding sequence of the secretion signal peptide is placed downstream of the coding sequence encoding the TPD polypeptide, hence the secretion signal peptide is placed at the C-terminus of the TPD polypeptide.

According to some embodiments of the invention, the secretion signal peptide is derived from a bacterial secretion apparatus (also called “machinery”) selected from the group consisting of Sec, Tat, T1SS, T2SS, T3SS, T4SS, T5SS, SecA2, Sortase, Injectosome, and T7SS.

According to some embodiments of the invention, the heterologous polynucleotide comprises more than one nucleic acid sequence encoding a secretion signal peptide (SSP) being translationally fused to the nucleic acid sequence encoding the TPD polypeptide. For example, the heterologous polynucleotide may comprise two distinct (not identical) secretion signal peptides which are placed in tandem.

For example, the combination of two SSPs can be from the Sec and T3SS secretion machineries (e.g., as described in Shigeaki Matsuda et al., 2019. “Export of a Vibrio parahaemolyticus toxin by the Sec and type III secretion machineries in tandem”. Nat. Microbiol. 4(5):781-788).

The secretion signal peptide can be a cleavable peptide, which is cleaved from the TPD polypeptide upon secretion from the pathogenic bacteria, or it can be a non-cleavable signal peptide, which forms part of the secreted protein (i.e., the signal peptide attached to the TPD polypeptide).

In some embodiments, where the signal peptide is not cleavable and hence forms part of the secreted protein, the length of the secreted protein (i.e., the protein comprising the signal peptide and the TPD polypeptide) should be selected such that it is capable of being secreted from the non-pathogenic bacteria. For example, when using Salmonella as the non-pathogenic bacteria, the secreted protein should not exceed about 60 kDa (kilodalton).

According to some embodiments of the invention, the secreted protein which comprises the signal peptide and the TPD polypeptide has a size which does not exceed 60 kDa, e.g., a size which does not exceed 55 kDa, a size which does not exceed 50 kDa, a size which does not exceed 49 kDa, a size which does not exceed 48 kDa, or a size which does not exceed 47 kDa.

According to some embodiments of the invention, the length of the secreted protein which comprises the signal peptide and the TPD polypeptide is less than 600 amino acids, e.g., less than 590 amino acids, less than 580 amino acids, less than 570 amino acids, less than 560 amino acids, less than 550 amino acids, less than 540 amino acids, less than 530 amino acids, less than 520 amino acids, less than 510 amino acids, less than 500 amino acids, less than 490 amino acids, less than 480 amino acids, less than 470 amino acids, less than 460 amino acids, less than 450 amino acids, less than 440 amino acids, less than 430 amino acids, or less than 420 amino acids.

According to specific embodiments of the invention, the length of the secreted protein which comprises the signal peptide and the TPD polypeptide is less than 500 amino acids, less than 490 amino acids, less than 480 amino acids, less than 470 amino acids, less than 460 amino acids, less than 450 amino acids, less than 440 amino acids, less than 430 amino acids, or less than 420 amino acids.

In some embodiments, the secretion signal peptide is a short peptide of about 10-40 amino acids.

Examples of secretion signal peptides which use T3SS apparatus include, but are not limited to, SSPH1 (e.g., SEQ ID NO:7), SseJ (e.g., SEQ ID NO:8), SptP (e.g., SEQ ID NO:9), SopD-2 (e.g., SEQ ID NO:10), GtgE (e.g., SEQ ID NO:11), and YopH (e.g., SEQ ID NO:12).

Examples of secretion signal peptides which use the T2SS apparatus include, but are not limited to, the E. coli heat-stable enterotoxin b (STb) (e.g., SEQ ID NO:13), putative T2SS protein J [Citrobacter rodentium ICC168] (e.g., SEQ ID NO:14), putative T2SS protein K [Citrobacter rodentium ICC168] (e.g., SEQ ID NO:15), putative T2SS protein D [Citrobacter rodentium ICC168] (e.g., SEQ ID NO:16), Type II secretion system F domain protein [Mycolicibacterium rhodesiae JS60] (e.g., SEQ ID NO:17), and type II secretion system protein [Comamonas testosteroni KF-1] (e.g., SEQ ID NO:18).

A non-limiting example of a secretion signal peptide which uses the T1SS apparatus is CvaC15 (e.g., SEQ ID NO:19).

According to some embodiments of the invention, the heterologous polynucleotide further comprises a nucleic acid sequence for membrane anchorage or presentation of the TPD.

In some embodiments, the bacteria displays the recombinantly produced TPD polypeptide on its surface using a bacterial surface display system. Examples of bacterial surface display systems include outer membrane protein systems (e.g., LamB, FhuA, Ompl, OmpA, OmpC, OmpT, cCPX derived from OmpX, OprF, and PgsA), surface appendage systems (e.g., F pillin, FimH, FimA, FliC, and FliD), lipoprotein systems (e.g., INP, Lpp-OmpA, PAL, Tat-dependent, and TraT), and virulence factor-based systems (e.g., AIDA-1, EacA, EstA, EspP, MSP1 a, and invasin). Exemplary surface display systems are described, for example, in van Bloois, E., et al., Trends in Biotechnology, 2011, 29:79-86, which is hereby incorporated by reference.

In some embodiments, the bacteria comprises a plurality of nucleic acid sequences that encode for multiple different polypeptides that can be expressed by the same bacterial cell.

The heterologous polynucleotide may further include a linker or a tag to facilitate detection or purification of the amino acid sequence translated by the heterologous polynucleotide.

For example, a coding sequence of a HIS-tag (e.g., SEQ ID NO:4) can be also included in the heterologous polynucleotide. For example, FIG. 5A schematically depicts a heterologous polynucleotide according to some embodiments of the invention in which coding sequence of the secretion signal peptide is translationally fused upstream of the TPD coding sequence, and the coding sequence for the HIS-tag is translationally fused downstream of the and the TPD coding sequence.

According to some embodiments of the invention, the composition-of-matter further comprises at least one cancer therapeutic.

According to some embodiments of the invention, the at least one cancer therapeutic is recombinantly expressed by the non-pathogenic bacteria.

According to some embodiments of the invention, the at least one cancer therapeutic is a polypeptide which can be secreted from or presented on the membrane of the non-pathogenic bacteria. Non-limiting examples of such polypeptides are described hereinunder.

The at least one cancer therapeutic polypeptide can be expressed from the same heterologous polynucleotide as a translationally fused polypeptide from a single promoter. Additionally or alternatively, the at least one cancer therapeutic polypeptide can be expressed from the same heterologous polynucleotide using a different promoter than the promoter used to express the TPD polypeptide. Additionally or alternatively, the at least one cancer therapeutic polypeptide can be expressed from an additional heterologous polynucleotide which is inserted into the same bacterial cell (e.g., by genomic integration of the heterologous polynucleotide).

Additionally or alternatively, the at least one cancer therapeutic polypeptide is comprised in a heterologous polynucleotide which is inserted into a second non-pathogenic bacteria.

According to some embodiments of the invention, the composition-of-matter comprises two distinct bacterial cells, each comprises a different heterologous polynucleotide.

According to some embodiments of the invention, the at least one cancer therapeutic is loaded into the bacteria prior to administration to a subject.

In some embodiments, the at least one cancer therapeutic is loaded into the bacteria by growing the bacteria in a medium that contains a high concentration (e.g., at least 1 mM) of the cancer therapeutic, which leads to either uptake of the cancer therapeutic during cell growth or binding of the cancer therapeutic to the outside of the bacteria. The cancer therapeutic can be taken up passively (e.g. by diffusion and/or partitioning into the lipophilic cell membrane) or actively through membrane channels or transporters. In some embodiments, drug loading is improved by adding additional substances to the growth medium that either increase uptake of the molecule of interest or prevent extrusion of the molecules after uptake by the bacterium (e.g., efflux pump inhibitors like Verapamil, Reserpine, Carsonic acid, or Piperine). In some embodiments, the bacteria is loaded with the cancer therapeutic by mixing the bacteria with the cancer therapeutic and then subjecting the mixture to electroporation, for example, as described in Sustarsic M., et al., Cell Biol., 2014, 142(1):113-24, which is hereby incorporated by reference. In some embodiments, the cells can also be treated with an efflux pump inhibitor (see above) after the electroporation to prevent extrusion of the loaded molecules.

According to some embodiments of the invention, the at least one cancer therapeutic is an anti-cancer antibody.

Table 1 hereinbelow provides a non-limiting list of anti-cancer antibodies which can be secreted from or presented on a membrane of the non-pathogenic bacteria of some embodiments of the invention.

TABLE 1
Table 1.

| Name | Type | Use | Name | Type | Use |
|---|---|---|---|---|---|
| 3F8 | mab | neuroblastoma | Lirilumab | mab | solid and |
| | | | | | hematological |
| | | | | | cancers |
| Abagovomab | mab | ovarian cancer | Lorvotuzumab | mab | cancer |
| | | | mertansine | | |
| Abituzumab | mab | cancer | Lumretuzumab | mab | cancer |
| Adecatumumab | mab | prostate and breast | Mapatumumab | mab | cancer |
| | | cancer | | | |
| Alacizumab pegol | F(ab′)₂ | cancer | Margetuximab | mab | breast cancer |
| Amatuximab | mab | cancer | Matuzumab | mab | colorectal, lung and |
| | | | | | stomach cancer |
| Amivantamab | BsAb | non-small cell lung | Mirvetuximab | mab | ovarian cancer |
| | | cancer | soravtansine | | |
| Anatumomab | Fab | non-small cell lung | Mitumomab | mab | small cell lung |
| mafenatox | | cancer | | | carcinoma |
| Andecaliximab | mab | gastric cancer or | Modotuximab | mab | cancer |
| | | gastroesophageal | | | |
| | | junction | | | |
| | | adenocarcinoma | | | |
| Anetumab | mab | cancer | Monalizumab | mab | rheumatoid arthritis, |
| ravtansine | | | | | gynecologic |
| | | | | | malignancies, and |
| | | | | | other cancers |
| Ascrinvacumab | mab | cancer | Mosunetuzumab | BiTE | follicular |
| | | | | | lymphoma |
| Atezolizumab | mab | cancer | Nacolomab tafenatox | Fab | colorectal cancer |
| Avelumab | mab | cancer | Naptumomab | Fab | non-small cell lung |
| | | | estafenatox | | carcinoma, renal cell |
| | | | | | carcinoma |
| Azintuxizumab | mab | cancer | Narnatumab | mab | cancer |
| vedotin | | | | | |
| BCD-100 | | melanoma | Navicixizumab | mab | cancer |
| Bemarituzumab | mab | gastric cancer or | Necitumumab | mab | non-small cell lung |
| | | gastroesophageal | | | carcinoma |
| | | junction | | | |
| | | adenocarcinoma | | | |
| Bermekimab | mab | colorectal cancer | Nesvacumab | mab | cancer |
| Bevacizumab | mab | metastatic | Nimotuzumab | mab | squamous cell |
| | | cancer, retinopathy | | | carcinoma, head |
| | | of prematurity | | | and neck cancer, |
| | | | | | nasopharyngeal |
| | | | | | cancer, glioma |
| Bivatuzumab | mab | squamous cell | Nivolumab | mab | cancer |
| | | carcinoma | | | |
| Brentuximab | mab | Hodgkin's | Ocaratuzumab | mab | cancer |
| vedotin | | lymphoma, | | | |
| | | anaplastic | | | |
| | | large-cell | | | |
| | | lymphoma | | | |
| Brontictuzumab | mab | cancer | Olaratumab | mab | cancer |
| Cabiralizumab | mab | metastatic | Oleclumab | mab | pancreatic and |
| | | pancreatic cancer | | | colorectal cancer |
| Camrelizumab | mab | hepatocellular | Omburtamab | mab | cancer |
| | | carcinoma | | | |
| Cantuzumab | mab | colorectal cancer | Onartuzumab | Fab | cancer |
| mertansine | | | | | |
| Cantuzumab | mab | cancers | Ontuxizumab | mab | cancer |
| ravtansine | | | | | |
| Capromab | mab | prostate cancer | Oportuzumab | scFv | bladder cancer |
| | | (detection) | monatox | | |
| Catumaxomab | 3funct | ovarian cancer, | Oregovomab | mab | ovarian cancer |
| | | malignant ascites, | | | |
| | | gastric cancer | | | |
| cBR96-doxorubicin | mab | cancer | Otlertuzumab | mab | cancer |
| immunoconjugate | | | | | |
| Cemiplimab | mab | cutaneous | Panitumumab | mab | colorectal cancer |
| | | squamous cell | | | |
| | | carcinoma | | | |
| Cergutuzumab | mab | cancer | Pankomab | mab | ovarian cancer |
| amunaleukin | | | | | |
| Cetrelimab | mab | cancer | Parsatuzumab | mab | cancer |
| Cetuximab | mab | metastatic | Pasotuxizumab | mab | cancer |
| | | colorectal cancer | | | |
| | | and head and | | | |
| | | neck cancer | | | |
| Cibisatamab | mab | cancer | Patritumab | mab | cancer |
| Citatuzumab | Fab | ovarian cancer | Pembrolizumab | mab | melanoma and other |
| bogatox | | and other solid | | | cancers |
| | | tumors | | | |
| Cixutumumab | mab | solid tumors | Pemtumomab | | cancer |
| Clivatuzumab | mab | pancreatic cancer | Pertuzumab | mab | cancer |
| tetraxetan | | | | | |
| Codrituzumab | mab | cancer | Pidilizumab | mab | cancer and |
| | | | | | infectious diseases |
| Cofetuzumab | mab | cancer | Pinatuzumab vedotin | mab | cancer |
| pelidotin | | | | | |
| Coltuximab | mab | cancer | Pritumumab | mab | brain cancer |
| ravtansine | | | | | |
| Conatumumab | mab | cancer | Racotumomab | mab | non-small cell lung |
| | | | | | cancer |
| Cusatuzumab | mab | cancer | Radretumab | mab | cancer |
| Dalotuzumab | mab | cancer | Ramucirumab | mab | solid tumors |
| Demcizumab | mab | cancer | Relatlimab | mab | melanoma |
| Denintuzumab | mab | cancer | Retifanlimab | mab | Merkel cell |
| mafodotin | | | | | carcinoma |
| Depatuxizumab | mab | glioblastoma | Rilotumumab | mab | solid tumors |
| mafodotin | | | | | |
| Derlotuximab biotin | mab | recurrent | Robatumumab | mab | cancer |
| | | glioblastoma | | | |
| | | multiforme | | | |
| Dinutuximab | mab | neuroblastoma | Rosmantuzumab | mab | cancer |
| Dinutuximab beta | mab | neuroblastoma | Rovalpituzumab | mab | small cell lung |
| | | | tesirine | | cancer |
| Dostarlimab | mab | endometrial cancer | Samalizumab | mab | cancer |
| Drozitumab | mab | cancer | Samrotamab vedotin | mab | cancer |
| DS-8201 | | gastric or | Satumomab pendetide | mab | cancer (diagnosis) |
| | | gastroesophageal | | | |
| | | junction | | | |
| | | adenocarcinoma | | | |
| Duligotuzumab | mab | testicular cancer | Seribantumab | mab | cancer |
| Durvalumab | mab | cancer | Sibrotuzumab | mab | cancer |
| Duvortuxizumab | scFv | cancer | Siltuximab | mab | cancer |
| Ecromeximab | mab | malignant | Sirtratumab vedotin | mab | cancer |
| | | melanoma | | | |
| Edrecolomab | mab | colorectal | Sofituzumab vedotin | mab | ovarian cancer |
| | | carcinoma | | | |
| Elgemtumab | mab | cancer | Solitomab | BiTE | gastrointestinal, |
| | | | | | lung, and other |
| | | | | | cancers |
| Emactuzumab | mab | cancer | Spartalizumab | mab | melanoma |
| Emibetuzumab | mab | cancer | Tacatuzumab | mab | cancer |
| | | | tetraxetan | | |
| Enapotamab vedotin | mab | cancer | Taplitumomab paptox | mab | cancer |
| Enavatuzumab | mab | cancer | Tarextumab | mab | cancer |
| Enfortumab vedotin | mab | urothelial cancer | Tavolimab | mab | cancer |
| Enoblituzumab | mab | cancer | Telisotuzumab | mab | cancer |
| Ensituximab | mab | cancer | Telisotuzumab | mab | cancer |
| | | | vedotin | | |
| Epratuzumab | mab | cancer, systemic | Tenatumomab | mab | cancer |
| | | lupus | | | |
| | | erythematosus | | | |
| | | (SLE) | | | |
| Ertumaxomab | 3funct | breast cancer | Tepoditamab | mab | cancer |
| Etaracizumab | mab | melanoma, prostate | Tetulomab | mab | cancer |
| | | cancer, ovarian | | | |
| | | cancer | | | |
| Farletuzumab | mab | ovarian cancer | Tigatuzumab | mab | cancer |
| Ficlatuzumab | mab | cancer | Timigutuzumab | mab | cancer |
| Figitumumab | mab | adrenocortical | Tiragotumab | mab | cancer |
| | | carcinoma, non- | | | |
| | | small cell lung | | | |
| | | carcinoma | | | |
| Flanvotumab | mab | melanoma | Tislelizumab | mab | non-small cell lung |
| | | | | | cancer |
| Futuximab | mab | cancer | Tisotumab vedotin | mab | relapsed or |
| | | | | | refractory cervical |
| | | | | | cancer[112][113] |
| Galcanezumab | mab | migraine | TNX-650 | | Hodgkin's lymphoma |
| Galiximab | mab | B-cell lymphoma | Tomuzotuximab | mab | cancer |
| Gancotamab | scFv | cancer | Tovetumab | mab | cancer |
| Ganitumab | mab | cancer | Trastuzumab | mab | breast cancer |
| Gatipotuzumab | mab | cancer | Trastuzumab | mab | breast cancer |
| | | | duocarmazine | | |
| Girentuximab | mab | clear cell renal cell | Trastuzumab | mab | breast cancer |
| | | carcinoma | emtansine | | |
| Glembatumumab | mab | melanoma, breast | TRBS07 | 3funct | melanoma |
| vedotin | | cancer | | | |
| Sintilimab | | squamous | Tremelimumab | mab | hepatocellular |
| | | cell non-small cell | | | carcinoma |
| | | lung cancer | | | |
| Icrucumab | mab | cancer | Tucotuzumab | mab | cancer |
| | | | celmoleukin | | |
| Ifabotuzumab | mab | glioblastoma | Urelumab | mab | cancer |
| | | multiforme | | | |
| Iladatuzumab | mab | cancer | Vandortuzumab | mab | cancer |
| vedotin | | | vedotin | | |
| Imalumab | mab | cancer | Vantictumab | mab | cancer |
| Imgatuzumab | mab | cancer | Vanucizumab | mab | cancer |
| Indatuximab | mab | cancer | Varlilumab | mab | solid tumors and |
| ravtansine | | | | | hematologic |
| | | | | | malignancies |
| Indusatumab | mab | cancer | Vesencumab | mab | solid malignancies |
| vedotin | | | | | |
| Inebilizumab | mab | cancer, systemic | Visilizumab | mab | Crohn's disease, |
| | | sclerosis, multiple | | | ulcerative colitis |
| | | sclerosis | | | |
| Intetumumab | mab | solid tumors | Vobarilizumab | scFv | inflammatory |
| | | (prostate cancer, | | | autoimmune |
| | | melanoma) | | | diseases |
| Ipilimumab | mab | melanoma | Volociximab | mab | solid tumors |
| Iratumumab | mab | Hodgkin's | Vonlerolizumab | mab | cancer |
| | | lymphoma | | | |
| Istiratumab | mab | advanced solid | Vopratelimab | mab | |
| | | tumors | | | |
| Labetuzumab | mab | colorectal cancer | Vorsetuzumab | mab | cancer |
| | | | mafodotin | | |
| Lacnotuzumab | mab | cancer | Votumumab | mab | colorectal tumors |
| Ladiratuzumab | mab | cancer | Zalutumumab | mab | squamous cell |
| vedotin | | | | | carcinoma of the |
| | | | | | head and neck |
| Leronlimab | mab | breast cancer, HIV | Zatuximab | mab | cancer |
| Lexatumumab | mab | cancer | Zenocutuzumab | mab | cancer |
| Lifastuzumab | mab | cancer | Zolbetuximab | mab | gastric cancer, |
| vedotin | | | | | gastrointestinal |
| | | | | | adenocarcinoma and |
| | | | | | pancreatic cancer |
| Losatuxizumab | mab | cancer | Lirilumab | mab | solid and |
| vedotin | | | | | hematological |
| | | | | | cancers |
| Lilotomab | mab | cancer | Lorvotuzumab | mab | cancer |
| satetraxetan | | | mertansine | | |
| Lintuzumab | mab | cancer | Lumretuzumab | mab | cancer |
| | | | Mapatumumab | mab | cancer |
| Gemtuzumab | mab | acute myelogenous | Tabalumab | mab | B-cell cancers |
| ozogamicin | | leukemia | | | |
| Tafasitamab | mab | relapsed or | Talacotuzumab | mab | leukemia |
| | | refractory diffuse | | | |
| | | large B-cell | | | |
| | | lymphoma | | | |

According to some embodiments of the invention the anti-cancer antibodies which can be secreted from or presented on a membrane of the non-pathogenic bacteria of some embodiments of the invention include, but are not limited to PD-1 inhibitors [e.g., Pembrolizumab (KEYTRUDA), Nivolumab (OPDIVO), Cemiplimab (LIBTAYO)], PD-L1 inhibitors [e.g., Atezolizumab (TECENTRIQ), Avelumab (BAVENCIO), Durvalumab (IMFINZI)], CTLA-4 inhibitors [e.g., Ipilimumab (YERVOY)], and TF inhibitors [e.g., tisotumab vedotin-tftv (TIVDAK)].

According to some embodiments of the invention, the at least one cancer therapeutic is a pro-domain of a protein selected from the group consisting of Matrix metalloproteinases (MMPs), A Disintegrin and Metalloproteinases (ADAMs), lysyl oxidases (LOX-family enzymes) and Meprin.

Non-limiting examples include, but are not limited to, A8P (ADAM8 Pro domain), LPD (LOX Pro Domain), Mpp (Meprin Pro Domain).

According to some embodiments of the invention, the at least one cancer therapeutic is LEM, an anti MMP14 Fab antibody.

According to some embodiments of the invention, the composition-of-matter of some embodiments of the invention comprises a combination of bacteria.

Table 2 provides non-limiting examples of combination of bacteria which can be used according to some embodiments of the invention.

TABLE 2
Table 2.
Combinations

STM-TPD + STM-A8P
STM-TPD + A8P
STM-TPD + STM-LPD
STM-TPD + LPD
STM-TPD + STM-Mpp
STM-TPD + Mpp
STM-TPD + STM-FabLEM
STM-TPD + FabLEM
STM-TPD + Reparixin
STM-TPD + α-PD1
STM-TPD + chemotherapy
STM-TPD + Herceptin
STM-TPD + 5FU

The composition-of-matter of some embodiments of the invention can be administered to an organism per se, or in a pharmaceutical composition where it is mixed with suitable carriers or excipients.

According to an aspect of some embodiments of the invention there is provided a pharmaceutical composition comprising a therapeutically effective amount of the composition-of-matter of some embodiments of the invention and a therapeutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the composition-of-matter of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections. For the CNS, the pharmaceutical composition can be delivered using neurosurgical strategies (e.g., intracerebral injection or intracerebroventricular infusion). Additionally or alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an acrosol spray presentation from a pressurized pack or a nebulizer with the use of a suitable propellant, e.g., dichlorodifluoromethane, trichlorofluoromethane, dichloro-tetrafluorocthane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (the composition-of-matter of some embodiments of the invention) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect of some embodiments of the invention there is provided a method of treating cancer in a subject, comprising administering to the subject a therapeutically effective amount of composition-of-matter of some embodiments of the invention or the pharmaceutical composition of some embodiments of the invention, thereby treating the subject.

According to some embodiments of the invention, the composition-of-matter or the pharmaceutical composition of some embodiments of the invention is for use in treating cancer.

The term “treating” refers to inhibiting, preventing or arresting the development of a pathology (e.g., cancer or cancer metastases) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology. Preferably, this term encompasses individuals who are at risk to develop the pathology.

According to some embodiments of the invention, the cancer is characterized by abnormal extracellular matrix (ECM) deposition and remodeling. For example, the TME may include abnormally high depositions of ECM, including collagen, as compared to a non-cancerous tissue.

According to some embodiments of the invention, the therapeutically effective amount of the composition-of-matter is selected capable of decreasing collagen levels in a tumor microenvironment.

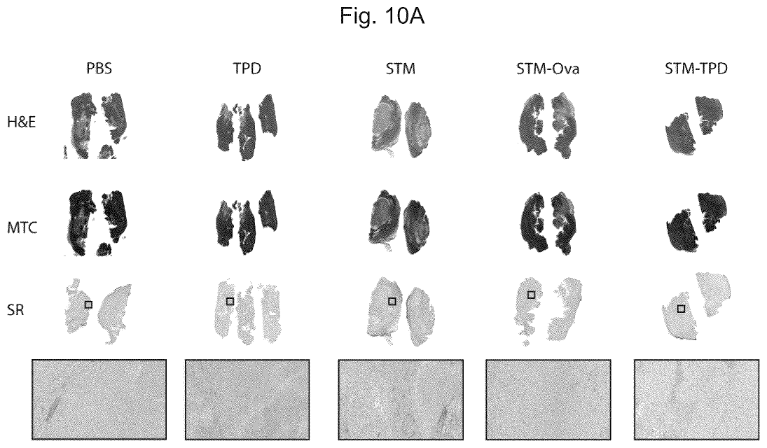

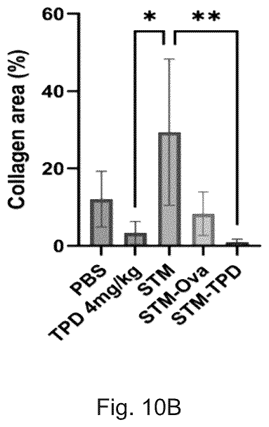

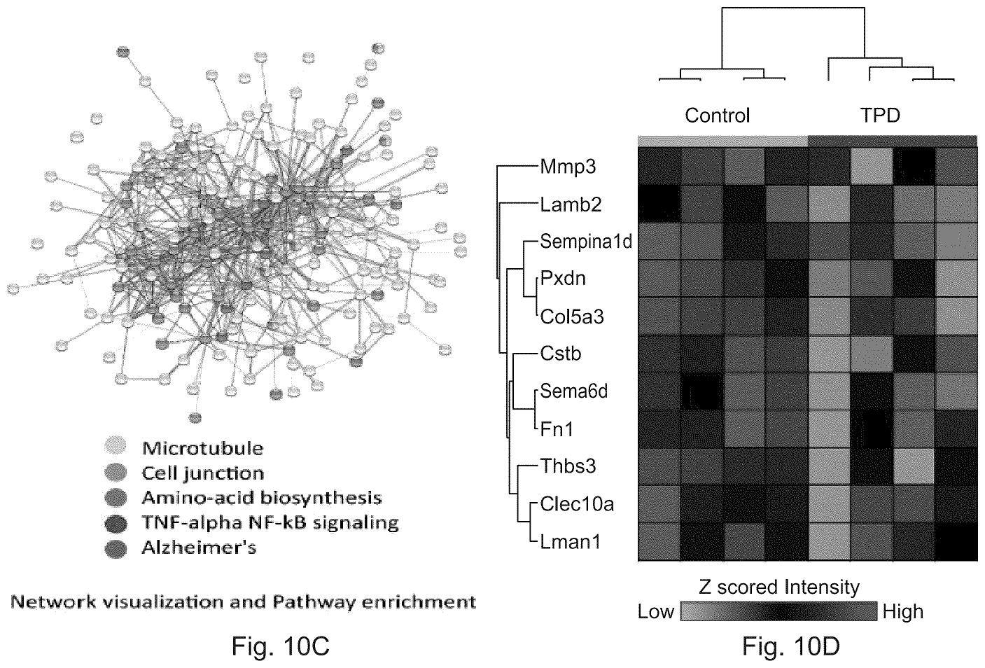

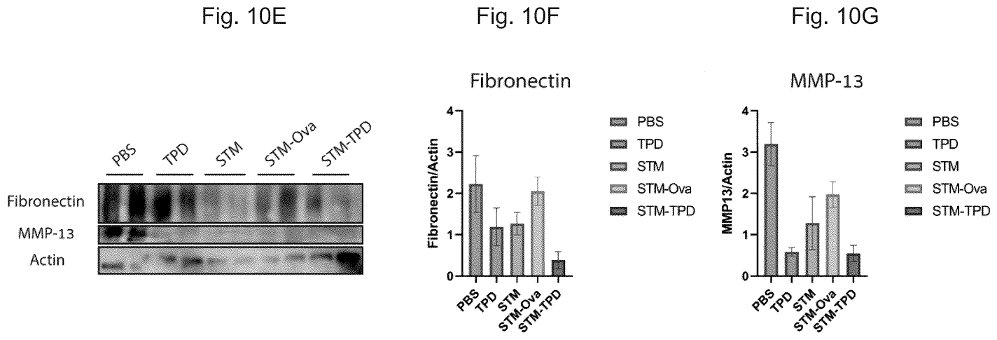

For example, FIG. 10B shows that in-vivo administration of the composition-of-matter of some embodiments of the invention to mice having LLC tumors resulted in significantly decreased collagen levels (STM-TPD) as compared to mice having the LLC tumors which were administered with a control composition-of-matter comprising the non-pathogenic bacteria yet being devoid of the TPD polypeptide (STM).

According to some embodiments of the invention, the therapeutically effective amount of the composition-of-matter of some embodiments of the invention is selected capable of decreasing at least one cancer characteristic selected from the group consisting of collagen levels in tumor microenvironment, tumor volume, tumor weight, cancer cell viability and cancer metastases as compared to a treatment regimen which does not comprise the composition-of-matter of some embodiments of the invention.

For example, the Examples section which follows shows that administration of more than 1*10⁶ CFU (colony forming unit) of the STM-TPD, e.g., about 3*10⁶ CFU resulted in significant reductions in tumor growth rate (FIG. 7F). It is noted that a high concentration of 6*10⁶ CFU of the STM-TPD was toxic to the animals.

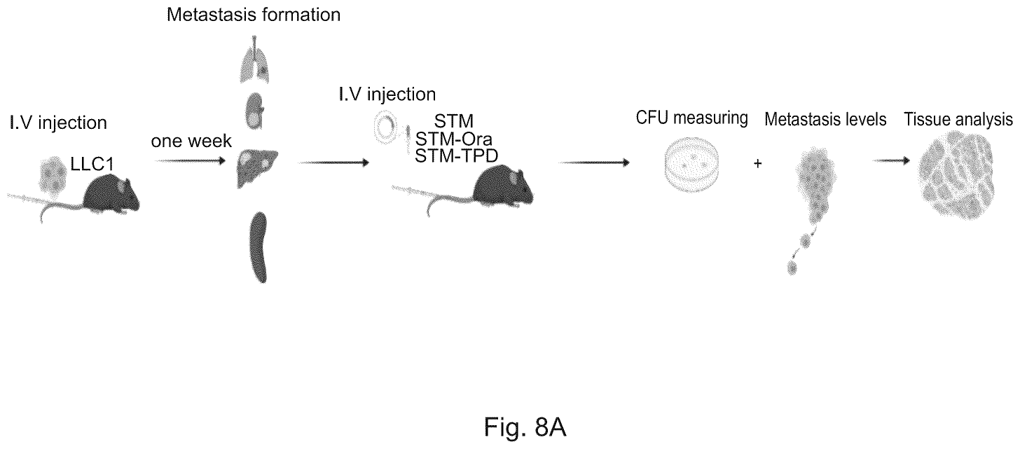

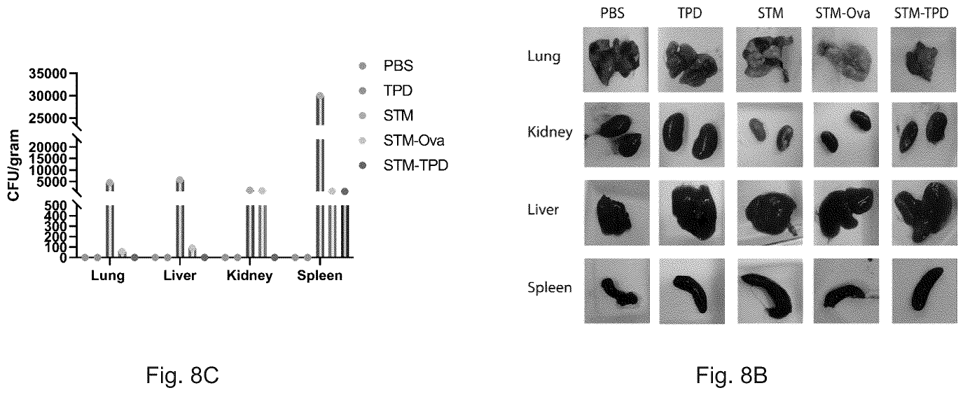

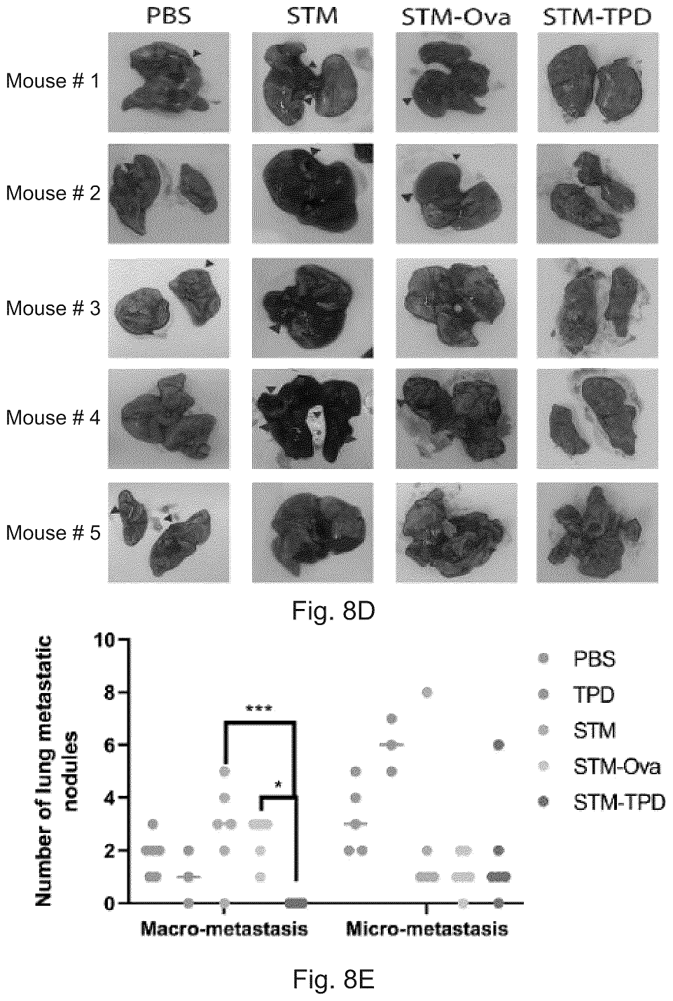

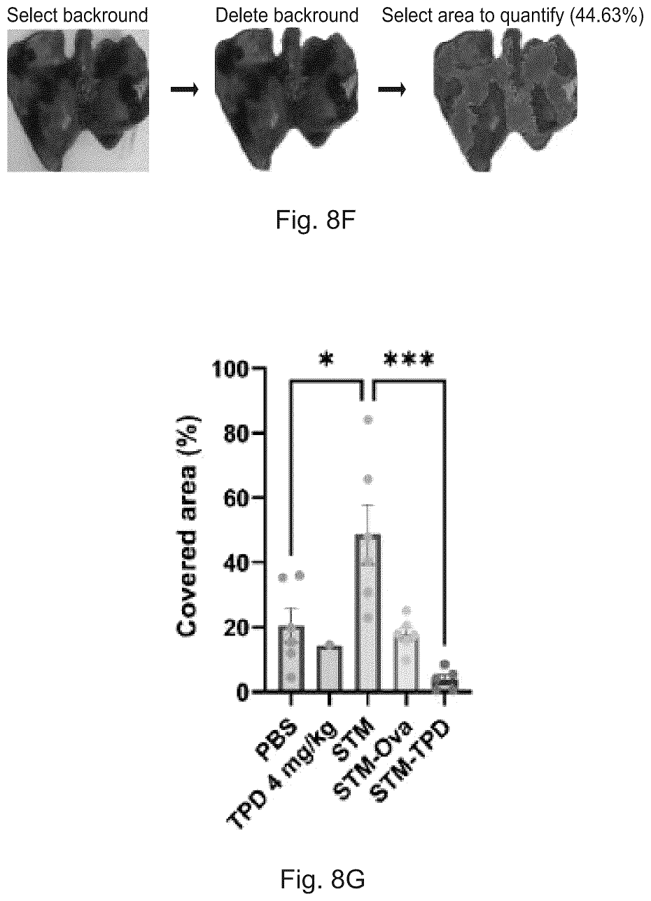

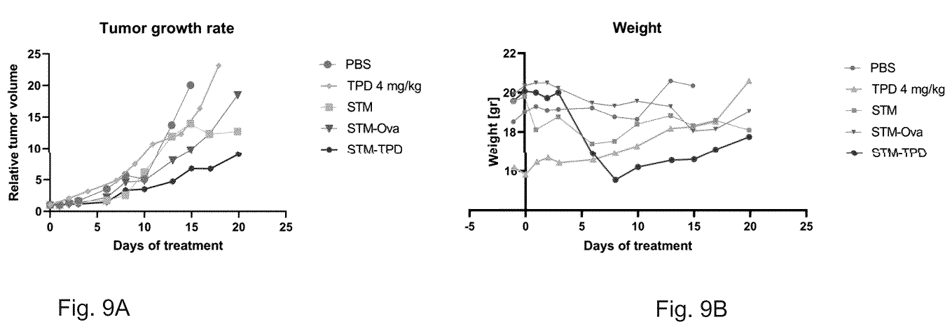

In addition, administration of 2*10⁶ CFU of the STM-TPD resulted in significantly decreased number of metastasis (e.g., macro-metastasis; FIG. 8E), decreased tumor volume (FIG. 9A) and decreased tumor weight (FIG. 9B).

According to some embodiments of the invention, the therapeutically effective amount of the composition-of-matter of some embodiments of the invention is selected capable of increasing survival of the subject as compared to a treatment regimen which does not comprise the composition-of-matter.

For example, the Examples section which follows shows that administration of more than 1*10⁶ CFU (colony forming unit) of the STM-TPD, e.g., about 3*10⁶ CFU increased survival of the treated animals (FIG. 7I).

According to some embodiments of the invention, the therapeutically effective amount of the composition-of-matter of some embodiments of the invention is between 10⁵ to 10¹⁰ CFU per kg of the subject.

The cancer which can be treated by the method of this aspect of some embodiments of the invention can be any solid tumor or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's discase, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with cosinophilia, B cell, basophilic, chronic myeloid, chronic, B cell, cosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissuc glial tumor, nervous tissue neuronal tumor, neurinoma, neuroblastoma, oligodendroglioma, ostcochondroma, ostcomyeloma, osteosarcoma (e.g., Ewing's), papilloma, transitional cell, phcochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to some embodiments of the invention, the cancer comprises a solid tumor.

According to some embodiments of the invention, the cancer comprises a non-solid tumor.

According to some embodiments of the invention, the cancer comprises cancer metastases.

According to some embodiments of the invention, the cancer comprises lung cancer.

It should be noted that the decrease in collagen levels in the TME following treatment with the composition-of-matter of some embodiments of the invention can increase accessibility of an anti-cancer drug(s) to the tumor and/or TME, and thus can improve efficiency of treatment.

According to some embodiments of the invention, the method further comprises administering to the subject an anti-cancer drug in combination with, concomitantly with, or following administration of the composition-of-matter of some embodiments of the invention or the pharmaceutical composition of some embodiments of the invention.

Thus, according to some embodiments of the invention there are provided methods of enhancing therapeutic treatment of a cancer.

Therapeutic regimen for treatment of cancer suitable for combination with the composition-of-matter of some embodiments of the invention or the pharmaceutical composition of some embodiments of the invention include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

The term “polypeptide” refers to a polymer of natural or synthetic amino acids, encompassing native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)—CO—), ester bonds (—C(═O)—O—), ketomethylene bonds (—CO—CH2—), sulfinylmethylene bonds (—S(═O)—CH2—), α-aza bonds (—NH—N(R)—CO—), wherein R is any alkyl (e.g., methyl), amine bonds (—CH2—NH—), sulfide bonds (—CH2—S—), ethylene bonds (—CH2—CH2—), hydroxyethylene bonds (—CH(OH)—CH2—), thioamide bonds (—CS—NH—), olefinic double bonds (—CH═CH—), fluorinated olefinic double bonds (—CF═CH—), retro amide bonds (—NH—CO—), peptide derivatives (—N(R)—CH2—CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Table 3 below lists non-conventional or modified amino acids (e.g., synthetic) which can be used with some embodiments of the invention.

TABLE 3
Table 3.

| Non-conventional amino acid | Code | Non-conventional amino acid | Code |
|---|---|---|---|
| ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α-methylbutyrate | Nmaabu |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl | Nmbc | 1,2,3,4-tetrahydroisoquinoline-3- | Tic |
| ethylamino)cyclopropane | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The polypeptide and/or peptides of some embodiments of the invention are preferably utilized in a linear form, although it will be appreciated that in cases where cyclicization does not severely interfere with peptide characteristics, cyclic forms of the peptide can also be utilized.

Since the present peptides are preferably utilized in therapeutics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO:1 is expressed in a DNA sequence format (e.g., reciting T for thymine), but it can refer to either a DNA sequence that corresponds to a TPD nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### General Materials and Experimental Methods

### Cells

LLC1—Lewis Lung Carcinoma cells are established from lung of C57/BL6 mouse (S. Bertram et al. 1980, Cancer Letters 11:63-73) and were obtained from ATCC (CRL-1642).

LLC1 Syngeneic Lung cancer model-The syngeneic lung cancer is done on 6-weeks old C57/BL6 female mice. The mice were inoculated subcutaneously with 0.25×10⁶ LLC1 cells into the right flank. The evaluation of tumor formation was performed on day 5. Tumor volume was measured with a caliper and calculated by the (X×Y²)/2 formula (Y is the smallest value). When the average tumor volume was 100 mm³, the mice were shuffled into different treatment groups normally distributed. 2*10⁶ CFU of the bacteria were injected twice intravenously (i.v) to mouse tail vain.

STM-TPD engineering—The TPD construct was prepared by cloning the nucleic acid sequence (SEQ ID NO:1) encoding TPD (SEQ ID NO:2) into the pQE60 plasmid, in which a type III secretion system of STM, sspH1 (SEQ ID NO:3) was cloned on the 3′-end of the TPD coding sequence (corresponding to the C-terminus of TPD amino acid sequence), and a HIS-tag coding sequence (SEQ ID NO:4) was cloned on the 5′-end of the TPD coding sequence, (corresponding to the N-terminus of TPD amino acid sequence) using Gibson Assembly Kit (Promega). The plasmid was transformed into STM-YS1646 cells using a bacteria electroporator (BioRad). Colonies were validated using Sanger Sequencing and Western Blot against anti-HIS antibody (Abcam ab18184).

Cell culturing—LLC1 cells were grown in Dulbecco's Modified Eagle Medium (DMEM) supplemented with 10% fetal bovine serum (FBS), 1% glutamine, and 1% penicillin/streptomycin in 5% CO₂.

Conditioned medium preparation—The STM-TPD, STM-Ova, STM were grown and cultured for 12 hours at 37° C. up to a concentration of 4*10⁸ STM/ml. Then, the supernatant was filtered through a 0.22 um pore filter, concentrated ten times and dialyzed with PBS by Amicon tube, 4,000×g for 10 minutes. The quantity of the secreted sspH1-TPD protein, which has been produced by a specific number of known bacteria within a certain time frame, has been unveiled by Western Blot intensity.

Metastasis quantification was performed by measuring the area of metastasis that covers organ.

TNFα release assay—J7 macrophages at concentration of 20,000 cell/well in 96 wells were seeded in DMEM for overnight. The cells were incubated for 3 hours with conditioned medium at a dose-dependent manner of estimated secreted-TPD concentration (0-16 μM). TNFα concentrations in the media were measured with mouse TNFα ELISA kit (R&D) according to manufacture instructions.

CellTiter-Glo assay—LLC1 cells were incubated with conditioned mediums for 16 hours. Later, 100 μl (microliter) CTG reagent (Promega, G7570) was mixed with LLC1 cells from the 96-well-treatment plate. The mix was incubated for 10 minutes at room temperature on the shaker, followed by luminescence measurement.

Flow cytometry and analysis antibodies—Biotinylated Protein-L and streptavidin PE were purchased from Genscript (Piscata, NJ). Approximately 5*10⁴ cells (gated on live lymphocytes) were analyzed. Cells were stained in a FACS buffer made of PBS, 0.5% BSA, and 0.02% sodium azide.

Cytokine release assays-For these assays, 1×10⁵ tumor cells and 1×10⁵ electroporated T cells were incubated in 200 μl volume in a 96-well plate. They were co-cultured for 24 hours. Cytokines secretion was measured using a commercially available ELISA kit for IFNγ and TNFα (R&D Systems).

### Example 1

### TPD Inhibits Viablity of Lung Cancer Cells and Reduces Protein Levels pf Phospho-ERK In-Vitro

### Experimental Results

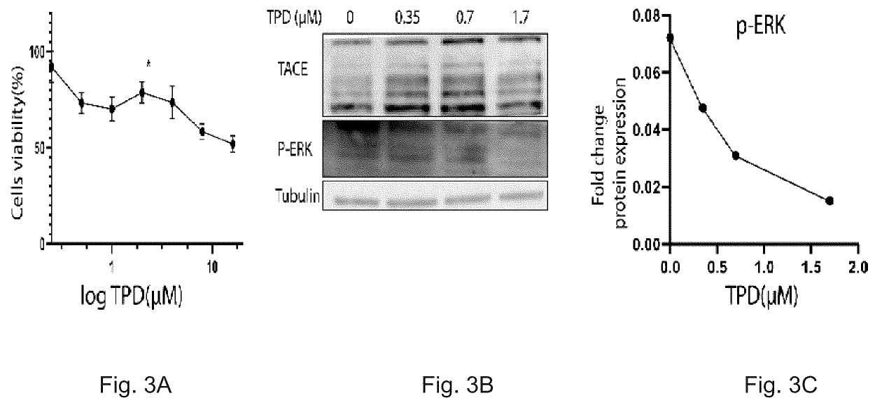

TPD inhibits the viability of lung cancer cells, and p-ERK protein levels—The luminescence-based cell viability was evaluated to determine the impact of TPD on LLC1 cells in-vitro. As illustrated in FIG. 3A, the viability of LLC-1 cells was notably diminished by TPD administration in a dose-dependent manner. To investigate TACE's mechanistic influence on IL-6 trans-signaling, the level of phospho-ERK, a downstream component, was analyzed. TPD treatment suppressed p-ERK in LLC-1 cells (FIGS. 3B-C). These findings strongly suggest that TPD inhibits the viability of KRAS-lung cancer cells and impedes p-ERK, pointing to the inhibition of IL-6 trans-signaling.

### Example 2

### Testing Ability of TPD to Treat Cancer In Vivo

### Experimental Results

Moderate effectiveness of TPD treatment in KRAS-mutant NSCLC and potential for improvement with bacterial delivery—To test the effect of TPD in treating cancer in vivo, the present inventors have employed mice bearing subcutaneous tumors of KRAS-mutant non-small cell lung cancer (NSCLC).

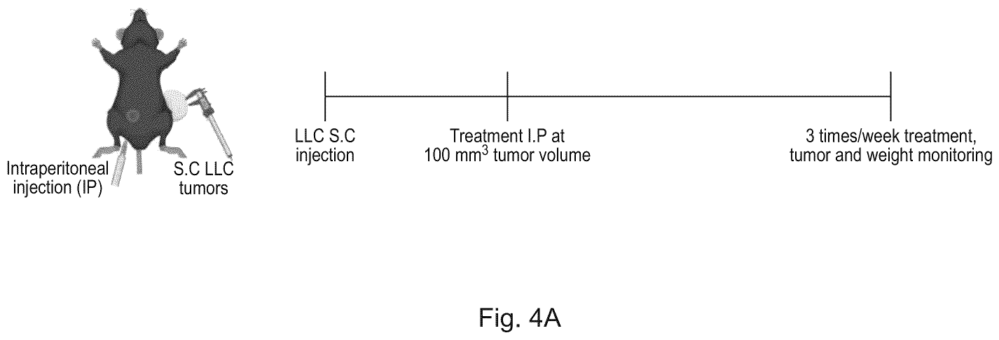

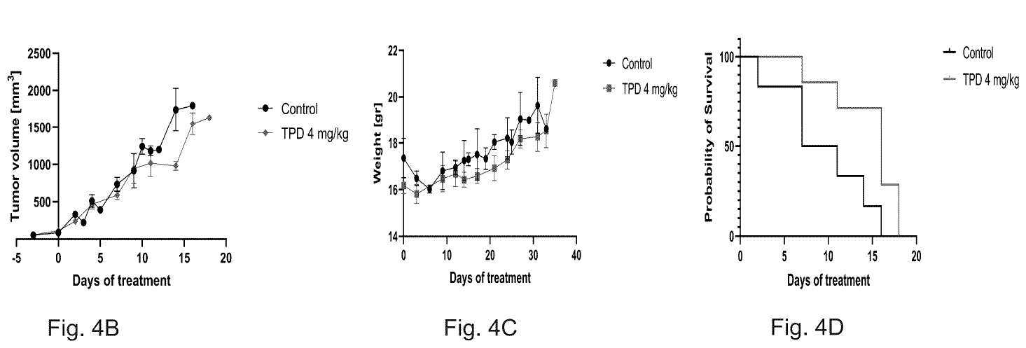

When tumors reached an approximate volume of 100 mm³ the treatment with isolated (e.g., pure) TPD commenced. Measurements of tumor volume started from day 7, and weight measurements were compared between TPD-treated and untreated mice (FIGS. 4B-C). The results revealed a modest efficiency of TPD treatment in prolonging the mice's survival 22 days following tumor implantation (FIG. 4D).

Without being bound to any theory, the present inventors have hypothesized that the TPD drug might have a limited penetration rate due to the high collagen content in these tumors. Furthermore, given the substantial presence of TACE in healthy tissues, TPD might have non-specific tissue targets.

### Example 3

### Generation of an Attenuated Salmonella which Secretes TPD

To overcome the moderate effect of TPD alone in inhibiting tumor growth the present inventors have engineered a recombinant attenuated salmonella (STM) to express the TPD polypeptide.

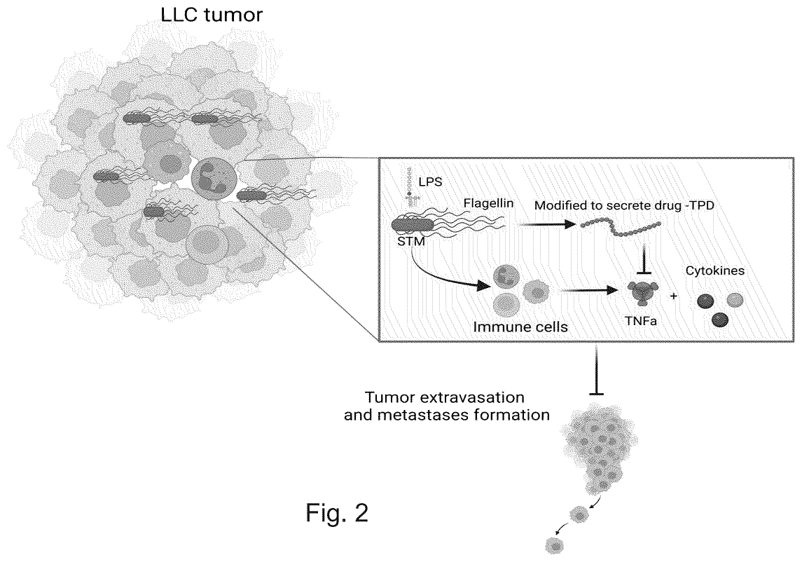

FIG. 2 schematically depicts a proposed mode of action according to some embodiments of the invention in which an attenuated salmonella secretes the TPD polypeptide.

### Experimental Results

Engineering TPD into STM secretion vectors for targeted delivery in the TME—the STM-TPD was designed for inhibiting the dysregulated activity of activated TACE on cell surfaces or within cells inside the tumor. The present inventors have utilized STM's tumor-tropism, anti-cancer activity, and immune activation properties to express and secrete the TPD. FIG. 5A schematically illustrates the construct according to some embodiments of the invention which is expressed in the attenuated salmonella. The present inventors have genetically modified the attenuated Salmonella STM-YS1646 (STM) strain to over-express and secrete TPD (STM-TPD). The TPD was engineered into the STM secretion vector using the TPD, a 200-amino acid folded protein domain. The secretion signal fused to the pro-domain and stabilizes the molecule, while the His tag serves as a reporter domain (FIG. 5A). Prediction of ssPH1-TPD (from STM) and TPD-HIS (from E. coli) structures using the AlphaFold deep learning algorithm provided valuable insights into the structural features of TPD (FIG. 5B). The TPD-containing colonies were validated using Sanger sequencing and Western Blot using against anti-HIS antibodies. The sspH1-TPD expression and secretion by STM-TPD were validated by secreted fraction separation, precipitation (trichloroacetic acid), and analysis by Western Blot against His-tag. A size-appropriate sspH1-TPD-His band at 46 kDa was detected in both bacterial lysate and secreted fraction (FIG. 5C-D). TPD-overexpression levels by STM were determined using densitometry analysis of recombinant TPD known amounts (FIG. 5E). The results of these experiments demonstrate the feasibility of engineering TPD into STM secretion vectors for targeted delivery in the TME.

### Example 4

### Characterization and Validation of Secreted TPD from the Engineered STM

### Experimental Results

Binding and inhibition efficiency of STM secreted TPD-Conditioned medium collected from culturing the engineered STM-TPD revealed the specificity of STM-TPD in its ability to inhibit the cleavage of TNFα induced by TACE on J7 cells (FIG. 6A). In addition, the STM-TPD conditioned medium inhibited lung cancer cell viability of the lung carcinoma cell line, LLC1, in cell-based assay in a dose-dependent manner (FIG. 6B), in comparison to unmodified STM (STM control) and generic 40 kDa peptide-STM (STM-Ova) (FIG. 6B). These results demonstrate the in-vitro inhibitory activity of the secreted protein. Overall, the results in FIGS. 6A-B provide evidence of STM-TPD as an inhibitor of TACE and cell viability in the TME.

### Example 5

### Specific Homing of the STM-TPD into Lung Cancer Tumors

### Experimental Results

In-vivo studies show that STM-TPD home specifically into LLC1 tumors while exhibiting low off target targeting to non-tumorigenic tissues—The selective accumulation of STM-TPD in the TME has been demonstrated in mice, with minimal off-target effects on non-tumorigenic tissues. Bacterial can specifically colonize the TME and avoid accumulation in healthy tissues, reducing the treatment-related side effects. In the current study, mice with sub-cutaneous (s.c) LLC1 tumors were given intravenous doses of 1×10⁵ and 1×10⁶ CFU of STM-TPD once, and the tumors were collected 10 days after bacteria injections. The amount of accumulated STM-TPD in tumors, livers, and spleens on the 10th day after infection (p.i.) was determined by CFU counting on ampicillin LB plates (FIG. 7B). The bacterial-tumor specificity was determined by CFU quantification using visual scoring and openCFU (FIGS. 7C and 7D). The results show that STM-TPD accumulated in the tumors, with minimal off-target effects in the liver, lung, and spleen. The effectiveness of STM-TPD treatment in LLC1 s.c tumors with minimal off-target effects was further demonstrated by the tumor growth rate and weight measurements taken under various STM-TPD doses and revealing that STM-TPD therapy significantly reduced tumor growth and weight (FIGS. 7D-H). The toxicity of STM-TPD was estimated by measuring mice survival under increased amounts of STM-TPD (FIG. 7I). These findings support the potential of STM-TPD as a targeted therapy for KRAS-mutant lung cancer.

### Example 6

### Antimetastatic Effects of the STM-TPD in Lung Cancer

### Experimental Results

STM-TPD as an Antimetastatic Agent in KRAS-Mutant Lung Cancer Mice—Since TACE involves several pro-cancer and pro-metastasis pathways, the present inventors assumed the STM-TPD would have an antimetastatic effect. LLC cells were injected I.V. into C57/BL6 mice. After one-week, metastatic niches were formed (FIG. 8A). Then, the mice were further injected I.V. with one of the following treatments: 2*10⁶ CFU of the STM-TPD, the unmodified STM, the STM-Ova, and 4 mg/kg TPD, or PBS (FIG. 8A). STM-TPD therapeutic administration demonstrated a significant reduction in metastasis spread of LLC cells (FIG. 8B) and accumulation of bacteria in the liver and spleen in KRAS-mutant lung cancer mouse models due to low metastatic niche. (FIG. 8C). These findings suggest the potential application of STM-TPD as an antimetastatic agent in cancer therapy.

After proving that the STM also gets to the initial metastatic niche, the present inventors performed a large cohort experiment and examined the effect of STM-TPD treatment on metastasis development. A significant reduction of LLC metastases development was shown in the lung under STM-TPD treatment. The decrease in lung metastasis was confirmed by visual scoring (FIG. 8E) and quantification (FIGS. 8F-G). The antimetastatic effect of STM-TPD is consistent with previous studies that show the crucial role of TACE in cancer metastasis and progression. These findings suggest that the STM-TPD is a promising therapeutic agent to reduce cancer metastasis in KRAS-mutant lung cancer.

### Example 7

### STM-TPD Treatment Inhibits Tumor Growth in the KRAS-Mutant Lung Cancer Model in Mice

### Experimental Results

The in-vivo inhibitory effect of STM-TPD on tumor progression is essential to evaluate its therapeutic potential. The present inventors used the KRAS-mutant lung cancer model, with subcutaneous injection of LLC. The injected mice were treated with 2*10⁶ CFU the STM-TPD and tumor growth was monitored. As shown in FIGS. 9A-B tumor growth was reduced in the STM-TPD treated mice as compared to control groups treated with PBS, recombinant TPD (4 mg/kg) STM-Ova or unmodified STM (2*10⁶ CFU) (FIG. 9A). The mice's weights were recorded while undergoing treatment (FIG. 9B). These findings suggest that STM-TPD has significant potential as a novel therapeutic agent for targeting dysregulated TACE activity and suppressing tumor growth in KRAS-mutant lung cancer models.

### Example 8

STM-TPD Treatment Reduces Collagen Deposition and Fibronectin in LLC Tumors

Collagen is a major extracellular matrix component. It plays a critical role in tumor progression and metastasis. Increased collagen deposition has been associated with enhanced tumor growth, invasion, and resistance to therapy. Inhibition of collagen deposition has been proposed as a promising strategy to limit tumor progression and metastasis.

### Experimental Results

To identify the effects of STM-TPD activity on ECM composition and its contribution to cancer inhibition, the present inventors have analyzed the LLC-S.C tumors. Ther results demonstrate that STM-TPD treatment leads to a reduction in collagen deposition and fibronectin in LLC tumors. The decrease in collagen deposition is supported by the histological images obtained from Hematoxylin and Eosin stain (H&E), Masson's trichrome (MTC), and Sirius Red (SR) staining, which show a reduction in collagen deposition in the STM-TPD treated group compared to the control groups; STM-Ova, unmodified STM, TPD, and PBS (FIG. 10A). The quantification of SR staining further confirms this result, as a significant decrease in collagen deposition was observed in the STM-TPD treated group compared to the control groups (FIG. 10B).

To find new TPD indirect targets on the TME of LLC mice, the present inventors have performed a Degradomics analysis. The results showed a significant reduction in fibronectin and collagen 5 in tumors treated with TPD compared to control groups (FIGS. 10C-D).

Fibronectin is an extracellular matrix protein involved in cell adhesion, growth, and differentiation, while collagen 5 is a fibrillar collagen that plays a role in tissue remodeling and repair. The reduction in fibronectin and collagen five levels suggests that TPD treatment may alter the TME and inhibit tumor growth by disrupting the extracellular matrix. The present inventors have further measured the effect of STM-TPD compared to TPD, STM, STM-Ova, and PBS by Western Blot of fibronectin and MMP13 and found that both are decreased under STM-TPD treatment (FIG. 10E-F). These findings support the potential of STM-TPD as a therapeutic agent by modulating the TME.

### Example 9

### STM-TPD System for Suppressing Tumor Growth and Modulating the TME Through Inhibition of TACE Dysregulation and Reduction of Pro-Tumorigenic Signaling

The novel bacterial system described herein (e.g., the bacterial system referred to as “STM-TPD”), has been engineered to secrete TPD. This innovative system has demonstrated the ability to block the aberrant behavior of TACE, consequently restraining the advancement of tumors both in-vitro and in-vivo (FIGS. 6A-D, 7A-I, 8A-F and 9A-B). Furthermore, STM-TPD treatment was shown to reduce the presence of collagen deposition and fibronectin within primary tumors (FIGS. 10A-G).

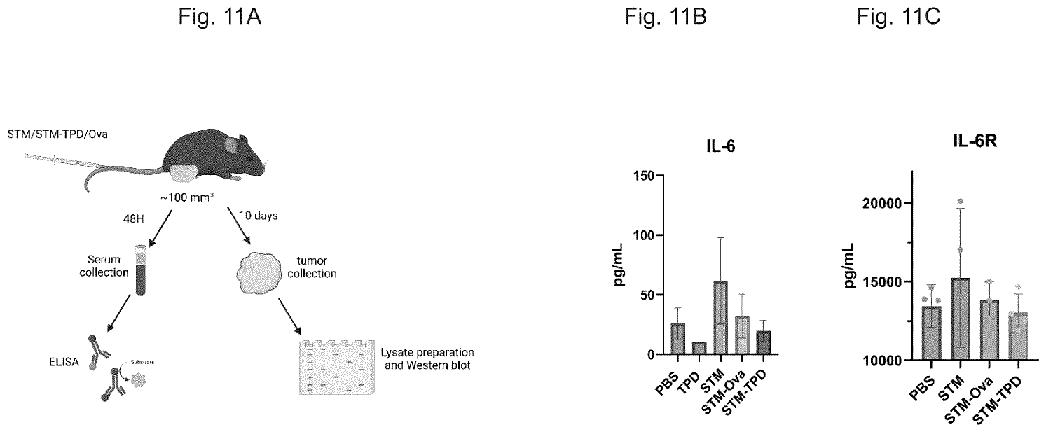

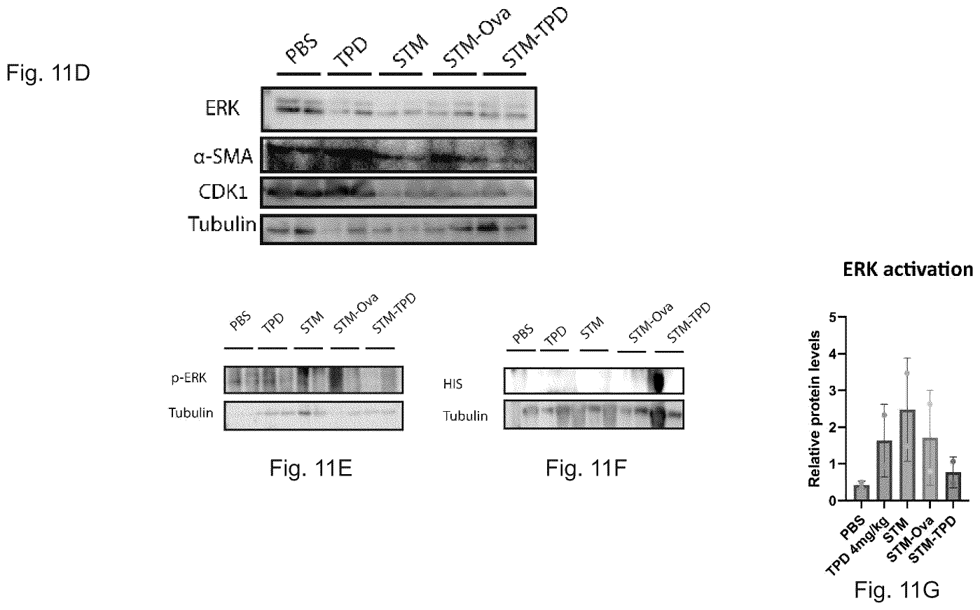

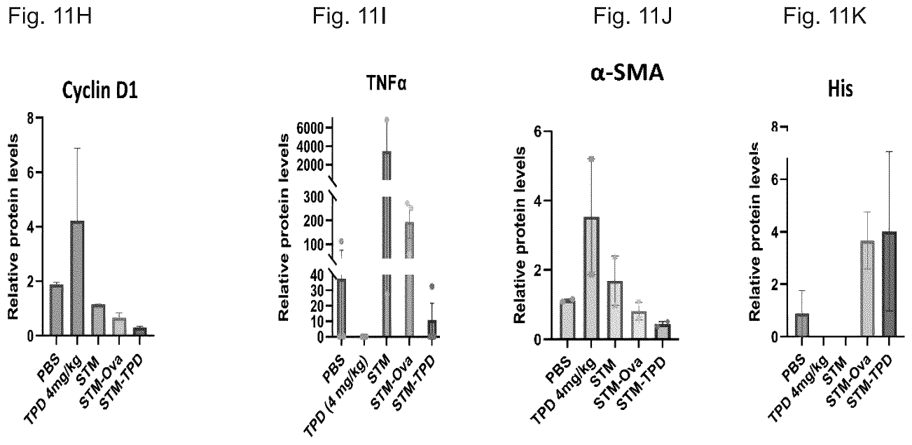

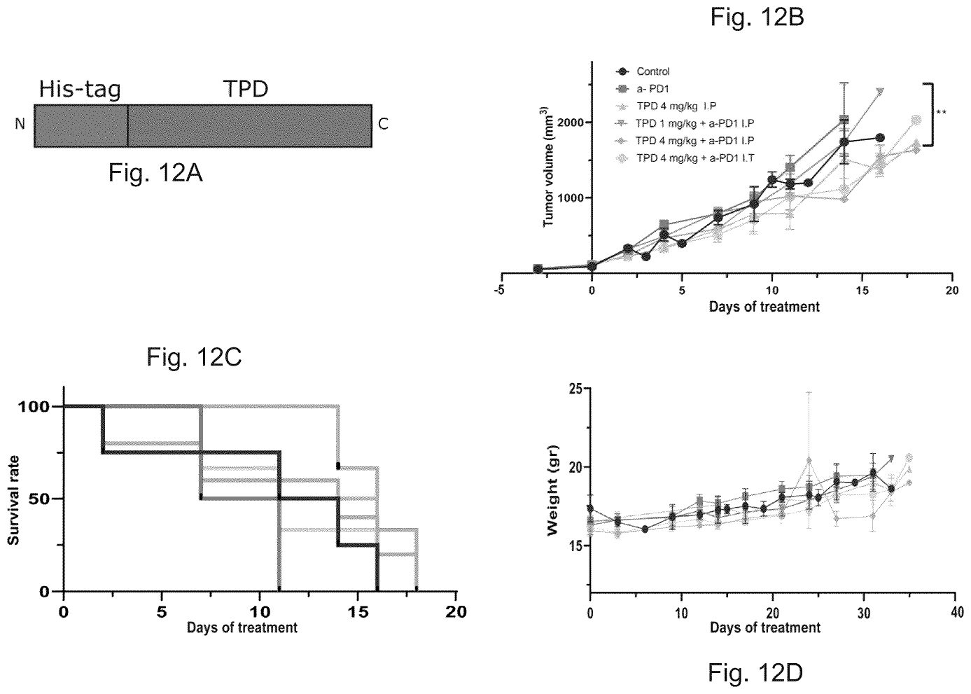

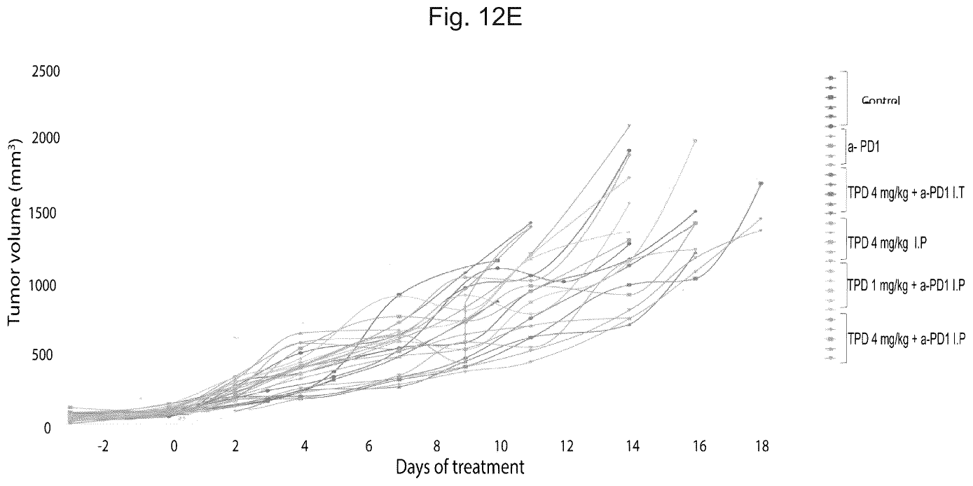

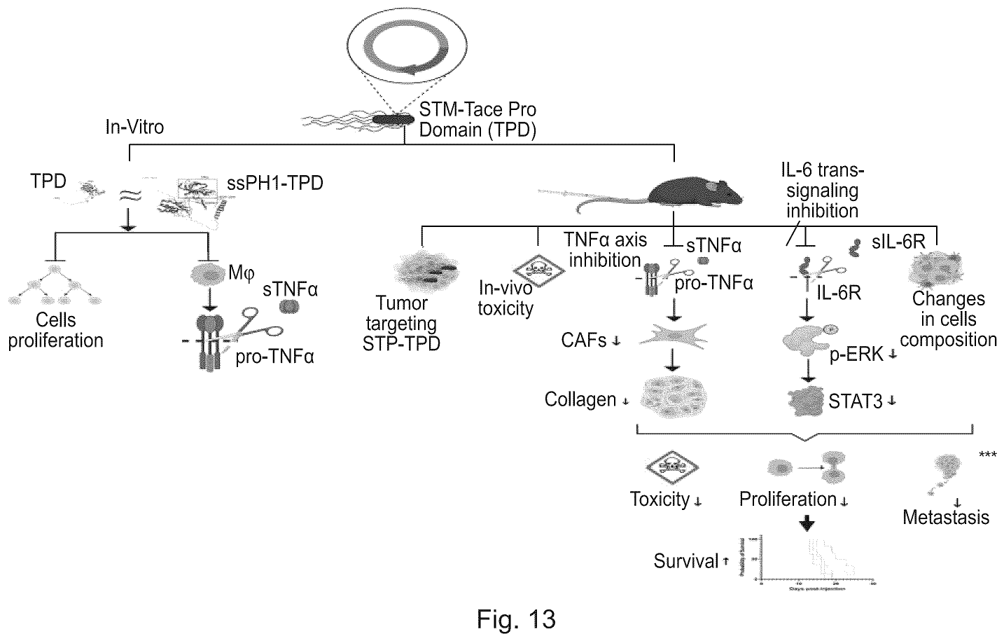

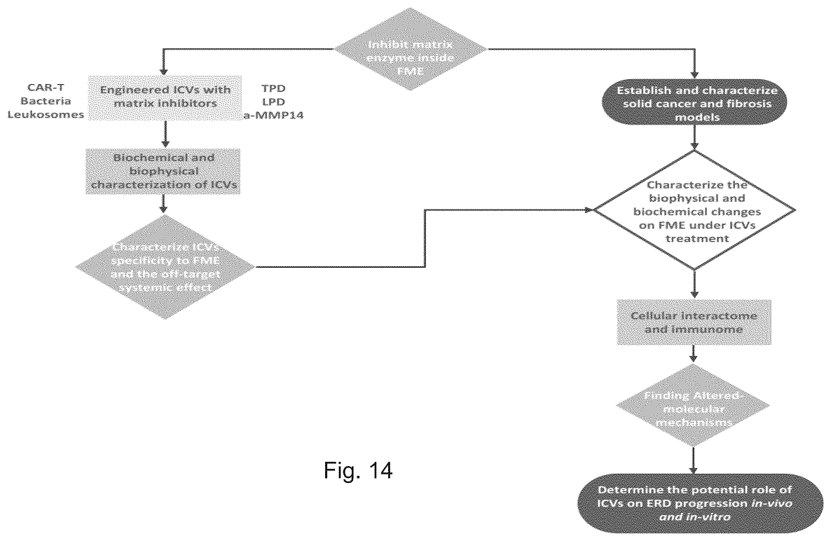

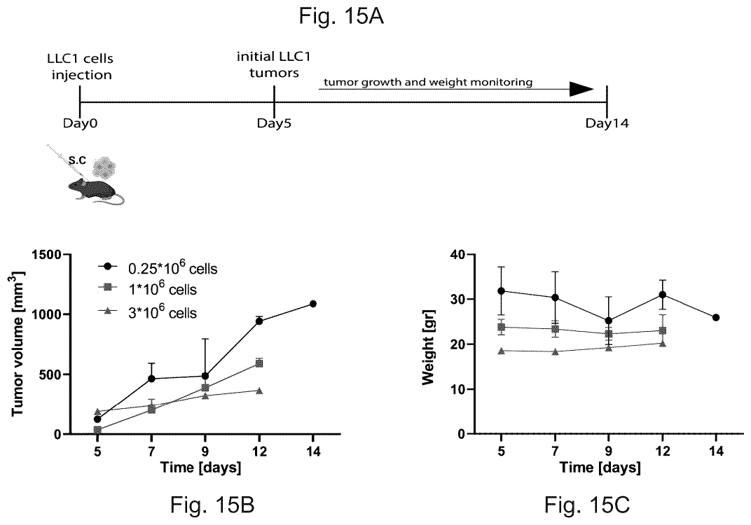

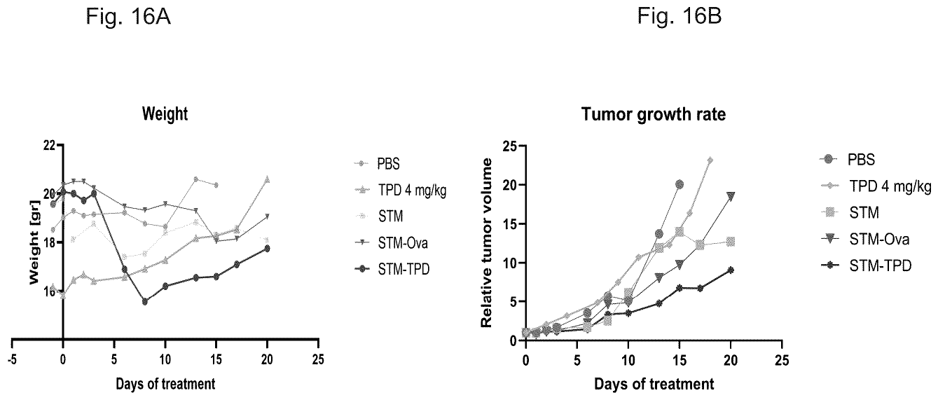

To further explore these effects, the present inventors conducted a protein analysis. This analysis assessed various TACE-associated signaling in LLC syngeneic mice, including IL-6 trans-signaling and TNFα. The mice were treated with STM, STM-TPD, TPD, STM-Ova, unmodified STM, or PBS. To achieve this, the present inventors have utilized Western blotting techniques on the primary tumors 10 days after bacteria injection and on blood serum 48 hours post-injection (schematically illustrated in FIG. 11A). ELISA assay performed on the blood serum of the mice demonstrated reduced levels of IL-6 and sIL-6R 48 hours after bacterial injection (FIGS. 11B-C). This suggests the potential for systemic effects triggered by STM-TPD treatment. In addition, the Western blot analysis highlighted diminished ERK activation in STM-TPD treated tumors as was measured by the p-ERK/ERK ratio (FIG. 11D-G). To confirm the presence of ssPH1-TPD within cancer, the present inventors have conducted a Western blot analysis using an anti-HIS antibody (FIG. 11F, and FIG. 11K). Furthermore, a reduction in Cyclin D1 (CDK1), a factor associated with cell cycle progression in cancer, was observed under STM treatment (FIGS. 11D, and 11H). Considering that TNFα serves as a critical substrate of TACE, the present inventors measured the levels of soluble TNFα (sTNFα) in the blood serum. The STM-TPD treated mice displayed decreased sTNFα levels (FIG. 11I). Without being bound by any theory, the decrease in collagen deposition observed under STM-TPD treatment (FIG. 10B) with the lowered sTNFα levels could potentially elucidate the marked reduction in Cancer-associated fibroblasts (CAFs) indicated by α-SMA staining. This reduction may indicate a decrease in CAFs (FIGS. 11D-J). This is particularly remarkable because the inflammation induced by sTNFα contributes to producing collagen-producing CAFs. STM-TPD could repair this process along with IL-6 trans signaling inhibition. Taken together, these findings suggest that STM-TPD holds the potential to regulate TACE activation while inhibiting several pro-inflammatory and pro-cancer signaling pathways.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### References (Additional References are Cited in Text)

1. Chizuka, A. et al. Difference between hematological malignancy and Solid tumor research articles published in four major medical journals. Leukemia Preprint at https://doi(dot)org/10.1038/sj.leu.2404369 (2006).
2. Peng, D. H. et al. Collagen promotes anti-PD-1/PD-L1 resistance in cancer through LAIR1-dependent CD8+ T cell exhaustion. Nat Commun 11:4520, (2020).
3. Joyce, J. A. & Fearon, D. T. T cell exclusion, immune privilege, and the tumor microenvironment. Science Preprint at https://doi(dot)org/10.1126/science.aaa6204 (2015).
4. Poltavets, V., Kochetkova, M., Pitson, S. M. & Samuel, M. S. The role of the extracellular matrix and its molecular and cellular regulators in cancer cell plasticity. Front Oncol 8, 1-19 (2018).
5. Gouarderes, S., Mingotaud, A. F., Vicendo, P. & Gibot, L. Vascular and extracellular matrix remodeling by physical approaches to improve drug delivery at the tumor site. Expert Opinion on Drug Delivery, 17:1703-1726 (2020).
6. Pickup, M. W., Mouw, J. K. & Weaver, V. M. The extracellular matrix modulates the hallmarks of cancer. EMBO Rep 15, 1243-1253 (2014).
7. Yang, B. et al. MicroRNA-3163 targets ADAM-17 and enhances the sensitivity of hepatocellular carcinoma cells to molecular targeted agents. Cell Death Dis 10, Article number: 784 (2019).
8. Jiao, X. et al. ADAM-17 is a poor prognostic indicator for patients with hilar cholangiocarcinoma and is regulated by FoxM1. BMC Cancer 18, Article number: 570 (2018).
9. Saad, M. I., Rose-John, S. & Jenkins, B. J. ADAM17: An emerging therapeutic target for lung cancer. Cancers (Basel) 11:1218, (2019).
10. Hurbin, A., Dubrez, L., Coll, J. L. & Favrot, M. C. Inhibition of apoptosis by amphiregulin via an insulin-like growth factor-1 receptor-dependent pathway in non-small cell lung cancer cell lines. Journal of Biological Chemistry 277, 49127-49133 (2002).
11. Jones, S. A. & Jenkins, B. J. Recent insights into targeting the IL-6 cytokine family in inflammatory diseases and cancer. Nat Rev Immunol 18, 773-789 (2018).
12. Miller, A. et al. Blockade of the IL-6 trans-signalling/STAT3 axis suppresses cachexia in Kras-induced lung adenocarcinoma. Oncogene 36, 3059-3066 (2017).
13. Borsellino, N., Bonavida, B. & Belldegrun, A. Endogenous Interleukin 6 Is a Resistance Factor for cis-Diamminedichloroplatinum and Etoposide-mediated Cytotoxicity of Human Prostate Carcinoma Cell Lines. Cancer Res 55, 4633-4639 (1995).
14. Garcia-Tuñón, I. et al. IL-6, its receptors and its relationship with bcl-2 and bax proteins in infiltrating and in situ human breast carcinoma. Histopathology 47, 82-89 (2005).
15. Hall, K. C. & Blobel, C. P. Interleukin-1 stimulates ADAM17 through a mechanism independent of its cytoplasmic domain or phosphorylation at threonine 735. PLoS One 7, (2012).
16. Armstrong, L., Godinho, S. I. H., Uppington, K. M., Whittington, H. A. & Millar, A. B. Contribution of TNF-α converting enzyme and proteinase-3 to TNF-α processing in human alveolar macrophages. Am J Respir Cell Mol Biol 34, 219-225 (2006).
17. Bolik, J. et al. Inhibition of ADAM17 impairs endothelial cell necroptosis and blocks metastasis. J. Exp. Med. 219(1), e20201039, (2021).
18. Wong, E. et al. Harnessing the natural inhibitory domain to control TNFα Converting Enzyme (TACE) activity in vivo. Sci Rep 6, 1-12 (2016).
19. Das, S. et al. ADAM17 Silencing in Mouse Colon Carcinoma Cells: The Effect on Tumoricidal Cytokines and Angiogenesis. PLoS One 7, (2012).
20. Meng, X. et al. ADAM17-siRNA inhibits MCF-7 breast cancer through EGFR-PI3K-AKT activation. Int J Oncol 49, 682-690 (2016).
21. Li, Y. et al. ADAM17 promotes cell migration and invasion through the integrin β1 pathway in hepatocellular carcinoma. Exp Cell Res 370, 373-382 (2018).
22. Mi, Z. et al. Salmonella-mediated cancer therapy: An innovative therapeutic strategy. J Cancer 10, 4765-4776 (2019).
23. Saad, M. I. et al. ADAM17 selectively activates the IL-6 trans-signaling/ERK MAPK axis in KRAS-addicted lung cancer. EMBO Mol Med 11(4):e9976, (2019).

What is claimed is:

1. A composition-of-matter comprising non-pathogenic bacteria capable of homing to a tumor, said bacteria comprising a heterologous polynucleotide comprising a nucleic acid sequence encoding a Pro Domain (TPD) polypeptide of TNFα Converting Enzyme (TACE), said TPD being devoid of a catalytic domain of said TACE and said TPD being secreted from or presented on a membrane of said bacteria.

2. The composition-of-matter of claim 1, wherein said non-pathogenic bacteria is capable of reducing IL6 signaling and ERK activation.

3. The composition-of-matter of claim 1, wherein said non-pathogenic bacteria is an attenuated Salmonella.

4. The composition-of-matter of claim 1, wherein said non-pathogenic bacteria is an attenuated pseudomonas aeruginosa (CHA-OST).

5. The composition-of-matter of claim 3, wherein said attenuated Salmonella is Salmonella Typhimurium strain VNP20009 (STM-YS1646).

6. The composition-of-matter of claim 1, wherein said non-pathogenic bacteria comprises modified lipopolysaccharides.

7. The composition-of-matter of claim 1, wherein said heterologous polynucleotide further comprises a nucleic acid sequence encoding a secretion signal peptide (SSP) being translationally fused to said nucleic acid sequence encoding said TPD polypeptide.

8. The composition-of-matter of claim 1, wherein said heterologous polynucleotide further comprises a nucleic acid sequence for membrane anchorage or presentation of said TPD.

9. The composition-of-matter of claim 1, wherein said TPD polypeptide comprises a modification which renders resistant of said TPD polypeptide to furin degradation.

10. The composition-of-matter of claim 9, wherein said modification is at a position selected from the group consisting of R58, R56, K57, R211, R214, and C184.

11. The composition-of-matter of claim 1, wherein said nucleic acid sequence encoding said TPD is operably linked to a constitutive promoter.

12. The composition-of-matter of claim 1, wherein said nucleic acid sequence encoding said TPD is operably linked to an inducible promoter specifically active under hypoxia.

13. The composition-of-matter of claim 1, wherein said non-pathogenic bacterium is capable of specifically proliferating under hypoxia conditions in a tumor microenvironment.

14. The composition-of-matter of claim 1, wherein said non-pathogenic bacteria is capable of specifically proliferating under necrosis in a tumor microenvironment.

15. The composition-of-matter of claim 1, further comprising at least one cancer therapeutic.

16. A pharmaceutical composition comprising a therapeutically effective amount of the composition-of-matter of claim 1, and a therapeutically acceptable carrier.

17. A method of treating cancer in a subject, comprising administering to the subject a therapeutically effective amount of composition-of-matter of claim 1, thereby treating the subject.

18. The method of claim 17, wherein said cancer comprises lung cancer.

19. The method of claim 17, wherein said cancer is characterized by abnormal extracellular matrix (ECM) deposition and remodeling.

20. The method of claim 17, wherein said therapeutically effective amount of said composition-of-matter is selected capable of decreasing collagen levels in a tumor microenvironment.
